(19) United States
(12) Patent Application Publication
(10) Pub. No.: US 2024/0148781 A1
(43) Pub. Date: May 9, 2024

(54) RHAMNAN SULFATE-BASED THERAPEUTICS FOR NONALCOHOLIC FATTY LIVER DISEASE AND CIRCADIAN RHYTHM DISORDERS

(71) Applicant: Board of Regents, The University of Texas System, Austin, TX (US)

(72) Inventors: Aaron Baker, Austin, TX (US); Nikita Patil, Austin, TX (US)

(21) Appl. No.: 18/501,504

(22) Filed: Nov. 3, 2023

Related U.S. Application Data
(60) Provisional application No. 63/382,347, filed on Nov. 4, 2022.

(51) Int. Cl. A61K 31/737; A61P 1/16

(52) U.S. Cl. CPC A61K 31/737; A61P 1/16

(57) ABSTRACT

The present invention describes compositions and methods for treatment of metabolic diseases and disorders, such as non-alcoholic fatty liver disease (NAFLD), and circadian rhythm-related disorders.

### CROSS-REFERENCE TO RELATED APPLICATIONS

This application claims priority under 35 U.S.C. § 119(e) to U.S. Provisional Application No. 63/382,347, filed Nov. 4, 2022, the contents of which are incorporated herein by reference in their entirety.

### STATEMENT REGARDING FEDERALLY SPONSORED RESEARCH OR DEVELOPMENT

This invention was made with government support under Grant no. R21 EB024147 awarded by the National Institutes of Health. The government has certain rights in the invention.

### BACKGROUND OF THE INVENTION

Cardiovascular disease continues to be a major cause of mortality worldwide, with roughly 17 million people dying from cardiovascular disease worldwide each year and an associated healthcare cost of over $500 billion in medical costs annually. Atherosclerosis is a chronic, progressive disease in which the accumulation of lipids, endothelial dysfunction and inflammatory processes within the vascular wall lead to plaque development (Stary, H. C., et al., 1994, Circulation, 89(5):2462-2478; Nielsen, L. B., 1996, Atherosclerosis, 123(1-2):1-15). Atherosclerotic plaques underlie a many forms of vascular disease and can lead to organ ischemia, stroke and myocardial infarction. Pharmacotherapy with statins have become a mainstay of modern treatment of atherosclerosis but often only slow the inevitable progression of the disease and do not represent a cure for the vast majority of patients (Bitzur, R., et al., 2013, Diabetes Care, 36(Supplement 2):5325-5330; Ward, N.C., et al., 2019, Circulation Research, 124(2):328-350). The endothelial glycocalyx is a layer of glycans that lines the interior of the artery and interacts with the flowing blood (Weinbaum, S., et al., 2007, Annual Review of Biomedical Engineering, 9:121-167).

The endothelial glycocalyx provides an atheroprotective effect within the artery, in part by maintaining the endothelial barrier function that prevents lipid deposition, reduces inflammation, and lowers the risk of arteriothombosis (Alphonsus, C. S., et al., 2014, Anaesthesia, 69(7):77-784; van den Berg, B. M., et al, 2003, Circulation Research, 92(6):592-594; Voyvodic, P. L., et al., 2014, Journal of Biological Chemistry, 289(14):9547-9559). During the progression of atherosclerotic disease, there is loss of the glycocalyx and associated lipid deposition, and inflammation (van den Bert, B. M., et al., 2009, Pflugers Archive: European Journal of Physiology, 457(6):1199-1206; Constantinescu, A. A., et al., 2003, Arteriosclerosis, Thrombosis, and Vascular Biology, 23(9):1541-1547; Mulivor, A. W., et al., 2004, American Journal of Physiology: Heart and Circulatory Physiology, 286(5):H1672-1680). Many of the major risk factors for atherosclerosis, including elevated C-reactive protein, hyperglycemia, oxidized lipids, and hyperlipidemia, lead to the depletion and degradation of the endothelial glycocalyx (Devaraj, S., et al., 2009, Cardiovascular Research, 84(3):479-484; Lopez-Quintero, S. V., et al., 2013, PLOS One, 8(11):e78954; Gouverneur, M., et al., 2006, Journal of Internal Medicine, 259(4):393-400; Rademakers, T., et al., 2013, Arteriosclerosis, Thrombosis, and Vascular Biology, 33(2):249-256; van den Berg, B. M., et al., 2006, Heart and Circulatory Physiology, 290(2):H915-920; Vink, H., et al., 2000, Circulation, 101(13):1500-1502). Major components of the glycocalyx include glycosaminoglycans, including heparan sulfate, chondroitin sulfate and hyaluronic acid. In the absence of these glycosaminoglycans, endothelial barrier function is compromised and immune cell adhesion is increased (Schmidt, E. P., et al., 2012, Nature Medicine, 18(8):1217-1223; Rehm, M., et al., 2004, Anesthesiology, 100(5):1211-1223; Mulivor, A. W., et al., 2002, American Journal of Physiology: Heart and Circulatory Physiology, 283(4):H1282-1291).

### SUMMARY OF THE INVENTION

In one aspect, the present disclosure provides a composition for treating or preventing a disease or disorder. In some embodiments, the composition is useful for treating or preventing metabolic diseases or disorders. In one embodiment, the compositions are useful for treating or preventing non-alcoholic fatty liver diseases (NAFLD). In one embodiment, the composition is useful for treating diseases or disorders associated with circadian rhythms. In one embodiment, the composition is useful for treating a work-shift disorder.

In another embodiment, the present invention provides a method of treating or preventing a disease or disorder. In some embodiments, the disease or disorder is a metabolic disease or disorder. In one embodiment, the metabolic disease or disorder is NAFLD. In some embodiments, the disease or disorder is associated with circadian rhythms. In one embodiment, the disorder associated with circadian rhythms is a work-shift disorder.

### BRIEF DESCRIPTION OF THE DRAWINGS

The following detailed description of various embodiments of the invention will be better understood when read in conjunction with the appended drawings. For the purpose of illustrating the invention, there are shown in the drawings, illustrative embodiments. It should be understood, however, that the invention is not limited to the precise arrangements and instrumentalities of the embodiments shown in the drawings.

FIG. 1A through FIG. 1J depict representative results demonstrating that vascular cells internalize rhamnan sulfate primarily through micropinocytosis. FIG. 1A depicts representative imaging of vascular smooth muscle cells (vSMCs) that were incubated with FITC-labeled rhamnan sulfate (RS) at 1 mg/mL with or without treatment with mitomycin for the indicated times. FIG. 1B depicts representative imaging of endothelial cells that were incubated with FITC-labeled RS at 1 mg/mL with or without treatment with rottlerin for the indicated times. FIG. 1C depicts a representative decrease in RS uptake in vSMCs treated with mitomycin occurring over three days. FIG. 1D depicts a representative increase in RS uptake in endothelial cells occurring over three days. FIG. 1E depicts representative quantification of total and nuclear uptake of RS in vSMCs treated with nystatin, an inhibitor of caveolin-mediated endocytosis. FIG. 1F depicts representative quantification of total and nuclear uptake in endothelial cells treated with nystatin. FIG. 1G depicts representative quantification of total and nuclear uptake of RS in vSMCs treated with Pitstop® 2, an inhibitor of clathrin-mediated endocytosis. FIG. 1H depicts representative quantification of total and nuclear uptake in endothelial cells treated with Pitstop® 2. FIG. 1I depicts representative quantification of total and nuclear uptake of RS in vSMCs treated with rottlerin, an inhibitor of macropinocytosis. FIG. 1J depicts representative quantification of total and nuclear uptake in endothelial cells treated with rottlerin. For FIGS. 1A and 1B, scale bar=20 μm. *, p<0.05 versus control day 1; †, p<0.05 versus treatment group on day 1; ‡, p<0.05 versus control for each time point; n=10 for all groups.

FIG. 2A through FIG. 2H depict representative results demonstrating decreases in proliferation and migration of vascular cells in response to RS treatment. FIG. 2A depicts a representative image of endothelial cells treated with FGF-2 and RS in an ORIS assay. FIG. 2B depicts representative results demonstrating migration of endothelial cells treated with FGF-2 is reduced by RS. FIG. 2C depicts representative results demonstrating that proliferation of endothelial cells treated with FGF-2 and RS was decreased after 72 hours. FIG. 2D depicts representative imaging of vSMCs expressing GFP that were treated with PDGF-BB and 0, 100, or 1000 μg/mL of RS in an ORIS assay. FIG. 2E depicts representative results demonstrating that migration of vSMCs treated with PDGF-BB is reduced by RS. FIG. 2F depicts representative results demonstrating that proliferation of vSMCs treated with PDGF-BB and RS was decreased after 72 hours. FIG. 2G depicts representative results demonstrating that migration of vSMCs treated with FGF-2 and RS increased after 72 hours of treatment. FIG. 2H depicts representative results demonstrating that proliferation of vSMCs by FGF-2 is reduced after 72 hours of treatment with RS. For FIGS. 2A and 2D, scale bar=100 μm. *, p<0.05 versus 0 μg/mL RS; †, p<0.05 versus 0 μg/mL RS+FGF-2/PDGF-BB; ‡, p<0.05 versus respective control; for FIGS. 2B, 2E, and 2G, n=10; for FIGS. 2C, 2F, and 2H, n=16.

FIG. 3A and FIG. 3B depict representative sensorgrams of RS. FIG. 3A depicts representative sensorgrams of RS and PDGF-BB. FIG. 3B depicts representative sensorgrams of RS and FGF-2. Dilutions of PDFG-BB (FIG. 3A) or FGF-2 (FIG. 3B) were introduced on a sensor surface with RS and the dissociation with HBS-EP buffer measured for three minutes. After three minutes, the surface was regenerated with 2 M NaCl and total response monitored. For FIGS. 3A and 3B, n=5.

FIG. 4A through FIG. 4F depict representative results demonstrating that RS reduces LDL permeability in endothelial cells. FIG. 4A depicts representative images of endothelial cells treated with RS (25 μg/mL) and heparinase III (125 mU/mL) and immunostained for heparan sulfate. Scale bar=20 μm. FIG. 4B depicts representative results demonstrating that heparan sulfate coverage, which is reduced by heparinase III, is increased in endothelial cells treated with RS. *, p<0.05 versus 0 mU/mL heparinase III; †, p<0.05 versus 0 mU/mL heparinase III+RS; ‡, p<0.05 versus 135 mU/mL heparinase III; n=8. FIG. 4C depicts representative results demonstrating that permeability of LDL is decreased in endothelial cells treated with RS regardless of heparinase III dose. n=4-8. FIG. 4D depicts representative results demonstrating that permeability of LDL is reduced in blank Transwell filters and those with subendothelial matrix or endothelial cell monolayer with RS incubation. n=4. FIG. 4E depicts representative results demonstrating that permeability of LDL is lower with RS treatment despite addition of TNF-α and cycloheximide (CHX). Heparin treatment produced no change in the same conditions. *, p<0.05 versus control; †, p<0.05 versus control+TNF-α/CHX; n=4-19. FIG. 4F depicts representative results demonstrating that permeability of VLDL is decreased by RS treatment. n=8. For FIGS. 4C, 4D, and 4F *, p<0.05 versus respective control.

FIG. 5A through FIG. 5K depict representative results demonstrating that RS reduces NF-κB activation in endothelial cells. FIG. 5A depicts a representative Western blot of endothelial cells treated with TNF-α (10 ng/mL) for 10 minutes to induce IκBα phosphorylation. FIG. 5B depicts representative quantification of Western blot analysis, as in FIG. 5A, showing a decrease in p-IκBα in total protein of cells pretreated with RS. n=2-15. FIG. 5C depicts a representative Western blot of endothelial cells treated with TNF-α (10 ng/mL) for 30 minutes to induce degradation if IκBα phosphorylation. FIG. 5D depicts representative quantification of Western blot analysis, as in FIG. 5C, demonstrating an increase in IκNα in the absence of TNF-α with RS treatment. n=7-11. FIG. 5E depicts a representative Western blot of endothelial cells treated with TNF-α for 30 minutes to cause NF-κB translocation into the nucleus. FIG. 5F depicts representative quantification of Western blot, as in FIG. 5E, demonstrating a decrease in the p65 subunit of NF-κB in the nuclear fraction of cells pre-treated with RS. n=6. FIG. 5G depicts a representative Western blot of endothelial cells treated with TNF-α (10 ng/mL) for 10 minutes to induce activation of IKK complexes. FIG. 5H depicts representative quantification of Western blot analysis, as in FIG. 5G, demonstrating a decrease in p-IKKα/β, but no change in IKKα and IKKβ in total protein of cells pretreated with RS. n=2-11. FIG. 5I depicts representative results of a TransAM assay, demonstrating reduced NF-κB/p65 activity after treatment with 100 μg/mL RS. n=8. FIG. 5J depicts representative sensorgrams of RS and NF-κB/p65. FIG. 5K depicts representative sensorgrams of RS and NF-κB/p50. For FIGS. 5J and 5K, dilutions of NF-κB/p65 or NF-κB/p50 were introduced on a sensor surface with RS and dissociation with HBS-EP buffer measured for three minutes. After three minutes, the surface was regenerated with 2 M NaCl and total response monitored. For FIGS. 5J and 5K, n=5. For FIGS. 5B, 5D, 5F, 5F, and 5I, *, p<0.05 versus control; †, p<0.05 versus TNF-α. For FIG. 5I, ‡, p<0.05 versus WT p65.

FIG. 6 depicts a representative Western blot analysis of the p65 subunit of NF-κB in the cytosolic fraction. Cytosolic protein is increased with 100 μg/mL RS but no change is observed with other treatments. †, p<0.05 versus TNF-α; n=8.

FIG. 7A through FIG. 7C depict representative pharmacokinetics of RS in vivo. FIG. 7A depicts a representative decrease in RS concentration in the abdominal aorta after 4 hours and accumulation in the thoracic aorta up to 24 hours after oral gavage. n=2. FIG. 7B depicts a representative increase in RS in total blood plasma up to 12 hours prior to decrease. In the liver an initial influx of RS up to 4 hours after oral gavage followed by steady accumulation after 12 hours. In the heart, RS concentration peaks at 4 hours after oral gavage. n=2. FIG. 7C depicts representative clearance of RS following an intravenous injection. The decay constant was calculated to be 0.0328 min⁻¹ for a half-life of 21.13 minutes. n=3.

FIG. 8 depicts a schematic representation of in vivo experiments for the prevention of atherosclerosis.

FIG. 9A through FIG. 9G depict representative reduction in atherosclerotic plaque area and plasma cholesterol in ApoE^{-/-} mice with RS treatment. FIG. 9A depicts a representative decrease in plasma cholesterol of 22.5% in female mice fed HFD and RS. FIG. 9B depicts a representative decrease in lipid deposition in the whole aorta of 45.2% in female and 36.4% in male ApoE^{-/-} mice fed HFD and RS. n=3. FIG. 9C depicts representative images of en face staining of the aortas of female and male mice for C57BL/6 mice fed a standard diet, ApoE^{-/-} mice fed a HFD, and ApoE^{-/-} mice fed a HFD and RS. FIG. 9D depicts representative imaging of aortic arch sections of female mice stained with Oil Red-O. FIG. 9E depicts a representative reduction in lipid deposition, lesion area, and stenosis in the aortic arches of female ApoE^{-/-} mice fed HFD with RS. Lipid deposition was reduced only in the aortic arches of males. n=7. FIG. 9F depicts representative imaging of histological sections of the thoracic aorta for female mice stained with Oil Red-O and hematoxylin and eosin. FIG. 9G depicts a representative reduction in lipid deposition, lesion area, and stenosis in the thoracic aortas of female ApoE^{-/-} mice fed a HFD with RS. n=7. For FIGS. 9A, 9B, 9E, and 9G, *, p<0.05 versus control; †, p<0.05 versus ApoE^{-/-} HFD. For FIGS. 9C, 9D, and 9F, scale bar=100 μm.

FIG. 10 depicts representative quantification of plasma triglyceride concentration of ApoE^{-/-} mice on HFD. Triglycerides increased in the plasma of female mice with HFD but no change is observed with RS treatment for either sex. n=10.

FIG. 11A through FIG. 11D depict representative body and tissue weight of ApoE^{-/-} mice on HFD and RS treatment. FIG. 11A depicts an average increase in body weight in both males and females on HFD over 13 weeks with no significant difference between HFD and HFD+RS groups. n=10. FIG. 11B depicts a representative lack of difference in mice on HFD with or without RS after 13 weeks. n=10. FIG. 11C depicts a representative lack of difference in the ratio of liver, gWAT, or iWAT weight to total body weight in female mice on HFD with or without RS after 13 weeks. FIG. 11D depicts a representative increase in the ratio of gWAT to total body weight in male mice on HFD+RS relative to HFD and a lack of increase in the ratio of liver or iWAT weight to total body weight. n=10. *, p<0.05 versus HFD.

FIG. 12A and FIG. 12B depict representative results demonstrating that adipocytes in white adipose tissue are not affected by RS treatment. FIG. 12A depicts representative histology sections of gWAT (top) and iWAT (bottom) from female mice stained with hematoxylin and eosin. FIG. 12B depicts representative histology sections of gWAT (top) and iWAT (bottom) from male mice stained with hematoxylin and eosin. No size of adipocytes was observed with HFD or HFFD+RS treatment. For FIGS. 12A and 12B, scale bar=200 μm; n=10.

FIG. 13 depicts a representative decrease in blood velocity as measured by ultrasound in ApoE^{-/-} mice treated with RS. After 12 weeks of HFD and RS, PSV, EDV, and MV are lower compared to no treatment in the ascending aortas of female mice while there is no change in blood velocity in aortic arches. Compared to no treatment, PSV and EDV are decreased in the descending aortas of female mice. In carotid arteries the PSV, EDV, and MV are lower compared to no treatment. In male mice there is no change in blood velocity in the ascending aorta while only EDV was lower compared to HFD mice in the aortic arches. In the descending aorta of male mice PSV, EDV, and MV are lower compared to no treatment. No change in blood velocity in the carotid arteries of male mice is observed. *, p<0.05 versus HFD; n=10.

FIG. 14A and FIG. 14B depict representative doppler ultrasound imaging used to measure blood velocity in the aortas of ApoE^{-/-} mice. FIG. 14A depicts representative images and velocity profiles of female ApoE^{-/-} mice fed HFD and HFD+RS. FIG. 14B depicts representative images and velocity profiles of male ApoE^{-/-} mice fed HFD and HFD+RS. For FIGS. 14A and 14B, n=10.

FIG. 15A and FIG. 15B depict representative doppler ultrasound imaging used to measure blood velocity in the left common carotid artery of ApoE^{-/-} mice. FIG. 15A depicts representative images and velocity profiles of female ApoE^{-/-} mice fed HFD and HFD+RS. FIG. 15B depicts representative images and velocity profiles of male ApoE^{-/-} mice fed HFD and HFD+RS. For FIGS. 15A and 15B, n=10.

FIG. 16 depicts representative quantification of elasticity of the aorta. Elasticity as measured by circumferential strain is higher in female ApoE^{-/-} mice treated with RS. n=10.

FIG. 17A through FIG. 17H depict representative attenuation of inflammatory markers in the aortic arch by RS. FIG. 17A depicts representative images of aortic arch histology sections of female mice stained for F4/80. FIG. 17B depicts a representative decrease in F4/80-positive staining in female mice treated with RS. FIG. 17C depicts representative imaging of aortic arch sections stained for IκBα. FIG. 17D depicts a representative increase in IκBα-positive staining in female ApoE^{-/-} mice fed HFD with RS. FIG. 17E depicts a representative decrease in NF-κB/p65 phosphorylation in female mice with RS treatment. FIG. 17F depicts a representative decrease in PECAM-1-positive staining in female and female mice treated with RS. FIG. 17G depicts a representative increase in eNOS staining in female mice treated with RS. FIG. 17H depicts a representative increase in α-SMA staining in female mice treated with RS. For FIGS. 17A and 17B, scale bar=100 μm. For FIGS. 17B and 17D-17H, *, p<0.05 versus control STD; †, p<0.05 versus ApoE^{-/-} HFD; n=7.

FIG. 18 depicts representative negative and positive controls of immunohistochemistry staining. Scale bar=100 μm.

FIG. 19 depicts representative immunohistochemical staining of aortic arches from female mice. For p-p65 and α-SMA, scale bar=20 μm. For eNOS and PECAM-1, scale bar=100 μm.

FIG. 20A depicts representative volcan plots of differential gene expression between the two specified groups. Genes that are significantly upregulated are shown in green and those significantly downregulated are shown in red. FIG. 20B depicts a representative Venn diagram of genes that are significantly upregulated in the groups. FIG. 20C depicts a representative Venn diagram of genes that are significantly downregulated in the groups. FIG. 20D depicts a representative clustering analysis of female mice with or without RS treatment. FIG. 20E depicts representative changes in individual genes that were in common for the groups. *, p<0.05 versus the respective non-RS treated group.

FIG. 21 depicts representative volcano plots for comparisons between the groups.

FIG. 22 depicts representative clustering analysis for the comparisons between the groups.

FIG. 23A and FIG. 23B depict representative gene ontology analysis. FIG. 23A depicts representative gene ontology analysis comparing HFD to HFD+RS male mice. FIG. 23B depicts representative gene ontology analysis comparing HFD to HFD+RS female mice.

FIG. 24A through FIG. 24H depict representative attenuation of lipid deposition in the liver with RS treatment in vivo. FIG. 24A depicts representative images of liver sections from female and male mice stained with hematoxylin and eosin. FIG. 24B depicts representative results demonstrating that RS led to reduced NAS scores in female livers compared to mice on HFD but did not alter the scores in male livers. n=10. FIG. 24C depicts representative images of liver sections from female and male mice stained with Oil Red-O. FIG. 24D depicts representative results demonstrating that RS decreased lipid deposition in the livers of female mice relative to mice on NFD but there was no change in the livers of male mice. FIG. 24E depicts representative Raman spectroscopy imaging of livers from female mice, demonstrating no difference in lipid unsaturation between HFD mice with or without RS. n=4. FIG. 24F depicts representative Raman spectroscopy imaging of livers from male mice, demonstrating a reduced degree of lipid unsaturation in the livers of mice treated with RS. n=3. FIG. 24G depicts representative average Raman spectra of livers from female mice. FIG. 24H depicts representative average Raman spectra of livers from male mice. For FIGS. 24A and 24B, scale bar=300 μm. For FIGS. 24E and 24F, scale bar=10 μm. *, p<0.05 versus control; t, p<0.05 versus ApoE^{-/-} HFD.

### DETAILED DESCRIPTION

The present disclosure provides compositions and methods for treating or preventing a disease or disorder. In some embodiments, the invention is directed to treating or preventing metabolic diseases or disorders. In one embodiment, the metabolic disease or disorder is non-alcoholic fatty liver disease (NAFLD).

In one embodiment, the invention relates to treating diseases or disorders associated with circadian rhythms. In one embodiment, the disease or disorder associated with circadian rhythms is a work-shift disorder.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The terms “biomarker” and “marker” are used herein interchangeably. They refer to a substance that is a distinctive indicator of a biological process, biological event and/or pathologic condition, disease or disorder.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

The term “detecting” or “detection,” means assessing the presence, absence, quantity or amount of a given substance (e.g., a reporter molecule) within a sample, including the derivation of qualitative or quantitative levels of such substances.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

A “therapeutic” treatment is a treatment administered to a subject who exhibits signs or symptoms of pathology disease or disorder, for the purpose of diminishing or eliminating those signs or symptoms.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell from a patient (e.g., for diagnosis or ex vivo applications), who has a disease or disorder contemplated herein, a sign or symptom of a disease or disorder contemplated herein or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder. The term “treating” or “treatment” also refers to a reduction in the severity of one or more symptoms by about 10%, about 20%, about 30%, about 40%, about 50%, about 60%, about 70%, about 80%, about 90% or about 100%.

As used herein, the terms “effective amount,” “pharmaceutically effective amount” and “therapeutically effective amount” refer to a sufficient amount of an agent to provide the desired biological or physiologic result. That result may be reduction and/or alleviation of a sign, a symptom, or a cause of a disease or disorder, or any other desired alteration of a biological system. An appropriate effective amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “potency” refers to the dose needed to produce half the maximal response (ED₅₀).

As used herein, the term “efficacy” refers to the maximal effect (Eₘₐₓ) achieved within an assay.

“Measuring” or “measurement,” or alternatively “detecting” or “detection,” means assessing the presence, absence, quantity or amount (which can be an effective amount) of either a given substance within a sample, including the derivation of qualitative or quantitative concentration levels of such substances, or otherwise evaluating the values or categorization of the substance or the sample.

As used herein, “associated” refers to coincidence with the development or manifestation of a disease, condition, or phenotype. Association may be due to, but is not limited to, genes responsible for housekeeping functions, those that are part of a pathway that is involved in a specific disease, condition, or phenotype and those that indirectly contribute to the manifestation of a disease, condition or phenotype.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “label” or a “detectable moiety” is a composition detectable by spectroscopic, photochemical, biochemical, immunochemical, chemical, magnetic resonance imaging, or other physical means.

As used herein, “nucleic acid” is meant to include any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). The term “nucleic acid” typically refers to large polynucleotides.

As used herein, the terms “peptide,” “polypeptide,” and “protein” are used interchangeably, and refer to a compound comprising amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein, is meant a molecule, such as a probe or aptamer, which recognizes and binds to another molecule or feature, but does not substantially recognize or bind other molecules or features in a sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based in part on the discovery that rhamnan sulfate inhibits lipid deposition in the artery and liver. Accordingly, in some embodiments, the invention is directed towards compositions comprising rhamnan sulfate for treating and preventing lipid deposition and associated diseases and disorders. In some embodiments, the disease or disorder associated with lipid deposition is a metabolic disease or disorder. In one embodiment, the metabolic disease or disorder is non-alcoholic fatty liver disease (NAFLD).

In other embodiments, the invention is directed towards methods of treating or preventing lipid deposition and associated diseases and disorders. In one embodiment, the method is for treating non-alcoholic fatty liver disease (NAFLD). In one embodiment, the method comprises administering to a subject in need a composition comprising rhamnan sulfate. In some embodiments, the method is for preventing progression of a disease or disorder. In some embodiments, the method is for preventing progression of NAFLD.

The present invention is also based in part on the discovery that rhamnan sulfate alters expression levels of circadian rhythm and circadian rhythm-related genes. In one embodiment, the invention is directed to compositions comprising rhamnan sulfate for treating diseases and disorders associated with misregulated circadian rhythm and/or circadian rhythm-related genes. In one embodiment, the composition is useful for treating a work-shift disorder.

In other embodiments, the invention is directed towards methods of treating or preventing diseases and disorders associated with misregulated circadian rhythm and/or circadian rhythm-related genes. In one embodiment, the method is for treating a work-shift disorder. In one embodiment, the method comprises administering to a subject in need a composition comprising rhamnan sulfate.

Compositions

In one aspect, the present disclosure provides a composition for treating and preventing lipid deposition. In some embodiments, the compositions are useful for treating or preventing diseases and disorders associated with lipid deposition. In one embodiment, the composition is useful for treating non-alcoholic fatty liver disease (NAFLD).

In another aspect, the disclosure provides a composition for preventing progression of diseases and disorders associated with lipid deposition. In some embodiments, the composition is useful for preventing progression of NAFLD. In some embodiments, the composition is useful for preventing NAFLD from progressing to liver fibrosis, non-alcoholic steatohepatitis (NASH), and/or cirrhosis.

In another embodiment, the disclosure provides a composition for treating and preventing a disease or disorder associated with misregulated circadian rhythm genes. In one embodiment, the disclosure provides a composition for treating and preventing a disease or disorder associated with misregulated circadian rhythm-related genes.

In one embodiment, the composition comprises rhamnan sulfate. In one embodiment, the composition comprises rhamnan sulfate that is largely or entirely of a specific molecular weight. In one embodiment, the rhamnan sulfate has an average molecular weight of between about 1 kDa, 2 kDa, 3 kDa, 4 kDa, 5 kDa, 6 kDa, 7 kDa, 8 kDa, 9 kDa, 10 kDa, 20 kDa, 40 kDa, 60 kDa, 80 kDa, 100 kDa, 110 kDa, 120 kDa, 130 kDa, 135 kDa, 140 kDa, 145 kDa, 150 kDa, 155 kDa, 160 kDa, 165 kDa, 170 kDa, 180 kDa, 190 kDa, or 200 kDa and about 100 kDa, 110 kDa, 120 kDa, 130 kDa, 135 kDa, 140 kDa, 145 kDa, 150 kDa, 155 kDa, 160 kDa, 165 kDa, 170 kDa, 180 kDa, 190 kDa, 200 kDa, 220 kDa, 240 kDa, 260 kDa, 280 kDa, or 300 kDa. In one embodiment, the rhamnan sulfate has a molecular weight of about 100-200 kDa, 110-190 kDa, 120-180 kDa, 130-170 kDa, 135-165 kDa, 140-160 kDa, 145-155 kDa, 146-154 kDa, 147-153 kDa, 148-152 kDa, or 148-151 kDa. In one embodiment, the rhamnan sulfate has an average molecular weight of about 150 kDa.

In one embodiment, the composition comprises more than one molecular weight or molecular weight range of rhamnan sulfate. In one embodiment, the composition comprises a first rhamnan sulfate and a second rhamnan sulfate. In one embodiment, the first rhamnan sulfate has an average molecular weight of between about 1 kDa, 2 kDa, 3 kDa, 4 kDa, 5 kDa, 6 kDa, 7 kDa, 8 kDa, 9 kDa, 10 kDa, 20 kDa, 40 kDa, 60 kDa, 80 kDa, 100 kDa, 110 kDa, 120 kDa, 130 kDa, 135 kDa, 140 kDa, 145 kDa, 150 kDa, 155 kDa, 160 kDa, 165 kDa, 170 kDa, 180 kDa, 190 kDa, or 200 kDa and about 100 kDa, 110 kDa, 120 kDa, 130 kDa, 135 kDa, 140 kDa, 145 kDa, 150 kDa, 155 kDa, 160 kDa, 165 kDa, 170 kDa, 180 kDa, 190 kDa, 200 kDa, 220 kDa, 240 kDa, 260 kDa, 280 kDa, or 300 kDa. In one embodiment, the first rhamnan sulfate has a molecular weight of about 100-200 kDa, 110-190 kDa, 120-180 kDa, 130-170 kDa, 135-165 kDa, 140-160 kDa, 145-155 kDa, 146-154 kDa, 147-153 kDa, 148-152 kDa, or 148-151 kDa. In one embodiment, the first rhamnan sulfate has an average molecular weight of about 150 kDa. In one embodiment, the second rhamnan sulfate has an average molecular weight of between about 1 kDa, 2 kDa, 3 kDa, 4 kDa, 5 kDa, 6 kDa, 7 kDa, 8 kDa, 9 kDa, 10 kDa, 20 kDa, 40 kDa, 60 kDa, 80 kDa, 100 kDa, 110 kDa, 120 kDa, 130 kDa, 135 kDa, 140 kDa, 145 kDa, 150 kDa, 155 kDa, 160 kDa, 165 kDa, 170 kDa, 180 kDa, 190 kDa, or kDa 200 kDa and about 100 kDa, 110 kDa, 120 kDa, 130 kDa, 135 kDa, 140 kDa, 145 kDa, 150 kDa, 155 kDa, 160 kDa, 165 kDa, 170 kDa, 180 kDa, 190 kDa, 200 kDa, 220 kDa, 240 kDa, 260 kDa, 280 kDa, or 300 kDa. In one embodiment, the second rhamnan sulfate has a molecular weight of about 100-200 kDa, 110-190 kDa, 120-180 kDa, 130-170 kDa, 135-165 kDa, 140-160 kDa, 145-155 kDa, 146-154 kDa, 147-153 kDa, 148-152 kDa, or 148-151 kDa.

In one embodiment, the weight ratio between the first rhamnan sulfate and the second rhamnan sulfate is about 1:1000 to 1000:1. In one embodiment, the weight ratio between the first rhamnan sulfate and the second rhamnan sulfate is from about 1:500 to about 500:1. In one embodiment, the weight ratio between the first rhamnan sulfate and the second rhamnan sulfate is from about 1:100 to about 100:1. In one embodiment, the weight ratio between the first rhamnan sulfate and the second rhamnan sulfate is from about 1:1 to about 50:1. In one embodiment, the weight ratio between the first rhamnan sulfate and the second rhamnan sulfate is from about 5:1 to about 25:1. In one embodiment, the weight ratio between the first rhamnan sulfate and the second rhamnan sulfate is about 1:1, about 1:10, about 1:15, about 1:20, about 1:30, about 1:40, about 1:50, about 1:60, about 1:70, about 1:80, about 1:90, about 1:100, about 1:125, about 1:150, about 1:175, about 1:200, about 1:250, about 1:300, about 1:350, about 1:400, about 1:450, about 1:500, about 1:550, about 1:600, about 1:650, about 1:700, about 1:750, about 190:1, about 1:800, about 1:850, about 1:900, about 1:950, or about 1:1000. In one embodiment, the weight ratio between the first rhamnan sulfate and the second rhamnan sulfate is about 2:1, about 3:1, about 4:1, about 5:1, about 8:1, about 10:1, about 15:1, about 20:1, about 25:1, about 30:1, about 40:1, about 50:1, about 60:1, about 70:1, about 80:1, about 90:1, about 100:1, about 125:1, about 150:1, about 175:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, or about 1000:1.

In some embodiments, the composition comprises one or more additional agents. Examples of agents include, but are not limited to, small molecules, nucleic acids, peptides, siRNAs, shRNAs, miRNAs, ribozymes, antisense nucleic acids, antagonists, inhibitors, agonists, partial agonists, inverse agonists, aptamers, peptidomimetics, viruses, bacteria, cells, or any combination thereof. In some embodiments, the one or more additional agents are one or more agents for treating a disease or disorder. In some embodiments, the one or more additional agents are one or more agents for treating a metabolic disease or disorder. In some embodiments, the one or more agents are one or more agents for treating NAFLD. Examples of additional agents for treating NAFLD include, but are not limited to, anti-cholesterol agents, anti-hypertriglyceridemia agents, anti-hypertensive agents, anti-diabetic agents, weight-loss agents, agents for treating cardiovascular disease, and agents for treating liver disease. In some embodiments, the one or more agents are one or more selected from the group consisting of saroglitazar, semaglutide, pioglitazone, vitamin E, metformin, ursodeoxycholic acid, dipeptidyl peptidase-4 inhibitors, statins, silymarin, and antiviral drugs.

In some embodiments, the one or more agents are one or more agents for treating a disease or disorder associated with mis-regulated circadian rhythm genes. In some embodiments, the one or more agents are one or more agents for treating a work-shift disorder. Examples of additional agents for treating work-shift disorder include, but are not limited to sleep promoting agents, wakefulness inducing agents, antidepressant agents, agent for treating ADHD, anxiolytic agents, antipsychotic agents, and antistress agents. In some embodiments, the one or more agents are one or more selected from the group consisting of melatonin, modafinil, armodafinil, tradizon, zolpidem, suvorexant, lemborexant, daridorexant, temazepam, amitriptyline, eszopiclone, quetiapine, mirtazapine, lorazepam, clonazepam, gabapentin, estazolam, doxepin, flurazepam, triazolam, diphenhydramine, ramelteon, zaleplon, doxylamine, quazepam, olanzapine, chloral hydrate, dimenhydrinate, pentobarbital, amobarbital, oxazepam, and phenobarbital.

### Pharmaceutical Compositions and Formulations

The invention also encompasses the use of pharmaceutical compositions to practice the methods of the invention. Such a pharmaceutical composition may consist of at least one composition of the invention or a salt thereof in a form suitable for administration to a subject, or the pharmaceutical composition may comprise at least one composition of the invention or a salt thereof, and one or more pharmaceutically acceptable carriers, one or more additional ingredients, or some combination of these. The rhamnan sulfate may be present in the pharmaceutical composition in the form of a physiologically acceptable salt, such as in combination with a physiologically acceptable cation or anion, as is well known in the art.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

Although the invention herein is principally directed to the ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions is contemplated include, but are not limited to, humans and other primates, mammals including commercially relevant mammals such as cattle, pigs, horses, sheep, cats, and dogs.

In one embodiment, the compositions utilized in the invention are formulated using one or more pharmaceutically acceptable excipients or carriers. In one embodiment, the pharmaceutical compositions comprise a therapeutically effective amount of a rhamnan sulfate and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In one embodiment isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition utilized in the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In one embodiment, the composition includes an anti-oxidant and a chelating agent that inhibits the degradation of the rhamnan sulfate. Exemplary antioxidants for some compounds are BHT, BHA, alpha-tocopherol and ascorbic acid in the range of about 0.01% to 0.3%. In one embodiment, the BHT is in the range of 0.03% to 0.1% by weight by total weight of the composition. In one embodiment, the chelating agent is present in an amount of from 0.01% to 0.5% by weight by total weight of the composition. Exemplary chelating agents include edetate salts (e.g. disodium edetate) and citric acid in the weight range of about 0.01% to 0.20%. In one embodiment, chelating agents may be in the range of 0.02% to 0.10% by weight by total weight of the composition. The chelating agent is useful for chelating metal ions in the composition that may be detrimental to the shelf life of the formulation. While BHT and disodium edetate are the exemplary antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition for use in the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the composition utilized in the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition for use in the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, such a mammal, including a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound for use in the invention is from about 1 and 5,000 mg/kg of body weight/per day. One of ordinary skill in the art would be able to study the relevant factors and make the determination regarding the effective amount of the therapeutic compound without undue experimentation.

The invention may be practiced as frequently as several times daily, or it may be practiced less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In certain embodiments, the composition of the present invention provides for a controlled release of a therapeutic agent. In certain instances, controlled- or sustained-release formulations of a pharmaceutical composition of the invention may be made using conventional technology, using for example proteins equipped with pH sensitive domains or protease-cleavable fragments. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, micro-particles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gel-caps, lozenges, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased subject compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In certain embodiments, the controlled-release formulation of the composition described herein allows for release of a therapeutic agent precisely when the agent is most needed. In another embodiment, the controlled-release formulation of the composition described herein allows for release of a therapeutic agent precisely in conditions in which the therapeutic agent is most active. In order to maintain this constant level of drug in the body, the drug must be released from the dosage form at a rate that will replace the amount of drug being metabolized and excreted from the body.

In certain embodiments, the composition provides for an environment-dependent release, when and where the therapeutic agent is triggered for release. For example, in certain embodiments the composition invention releases at least one therapeutic agent when and where the at least one therapeutic agent is needed. The triggering of release may be accomplished by a variety of factors within the microenvironment of the treatment or prevention site, including, but not limited to, temperature, pH, the presence or activity of a specific molecule or biomolecule, and the like.

Controlled-release of an active ingredient can be stimulated by various inducers, for example pH, temperature, enzymes, water or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation. In one embodiment of the invention, the compositions are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

In one embodiment, the invention is practiced in dosages that range from one to five times per day or more. In another embodiment, the invention is practiced in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Routes of administration of include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Methods

In some embodiments, the disclosure provides methods of treating and preventing a metabolic disorder. In one embodiment, the method treats and/or prevents NAFLD.

In some embodiments, the disclosure provides methods of treating and preventing a disease or disorder associated with misregulated circadian rhythm or circadian rhythm-related genes. In one embodiment, the method treats and/or prevents a work-shift disorder.

In some embodiments, the disclosure provides methods comprising administering to the subject a therapeutically effective amount of a composition comprising one or more rhamnan sulfate or a pharmaceutically acceptable salt or hydrate thereof. In one embodiment, the subject has a metabolic disorder, such as NAFLD. In one embodiment, the subject has a disease or disorder associated with one or more misregulated circadian rhythm or circadian rhythm-related genes. In one embodiment, the one or more circadian rhythm or circadian rhythm-related genes include one or more selected from the group consisting of PER3, TPPP, DBP, and WEE1.

In one embodiment, the disclosure provides methods comprising treating or preventing the effects of sleep disturbances on NAFLD, liver diseases, and vascular diseases by administering to a subject a composition comprising one or more rhamnan sulfate or a pharmaceutically acceptable salt or hydrate thereof. In one embodiment, the disclosure provides methods of treating or preventing the effects of sleep disorders on NAFLD, liver diseases, and vascular diseases by administering to a subject a composition comprising one or more rhamnan sulfate or a pharmaceutically acceptable salt or hydrate thereof. In one embodiment, the disclosure provides methods of treating or preventing NAFLD comprising modifying circadian rhythm pathways by administering to a subject a composition comprising one or more rhamnan sulfate or a pharmaceutically acceptable salt or hydrate thereof. In one embodiment, modifying circadian rhythm pathways comprises modifying one or more circadian rhythm or circadian rhythm-related genes selected from the group consisting of PER3, TPPP, DBP, and WEE1. In one embodiment, modifying the one or more circadian rhythm or circadian rhythm-related genes comprises administering to the subject a therapeutically effective amount of a composition comprising one or more rhamnan sulfate or a pharmaceutically acceptable salt or hydrate thereof.

In one aspect, the disclosure provides methods of treating or preventing the negative health effects associated with sleep deprivation by administering to a subject a composition comprising one or more rhamnan sulfate or a pharmaceutically acceptable salt or hydrate thereof. In one embodiment, the sleep deprivation results from one or more selected from the group consisting of sleep disorders, depression, anxiety, traumatic brain injury, post-traumatic stress disorder, work-shift disorders, shift work disorder, insomnia, and neurological disorders.

The methods of the invention thus encompass the use of pharmaceutical compositions comprising rhamnan sulfate to practice the methods of the invention. The pharmaceutical compositions useful for practicing the invention may be administered to deliver a dose of from 1 ng/kg/day and 1,000 mg/kg/day. In one embodiment, the invention envisions administration of a dose which results in a concentration of the compound of the present invention from 1 μM and 100 μM in a mammal.

Typically, dosages which may be administered in a method of the invention to a mammal, such as a human, range in amount from 0.5 μg to about 5,000 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 1 μg to about 750 mg per kilogram of body weight of the mammal. In some embodiments, the dosage of the compound is about 750 mg per kilogram of body weight of the mammal. In some embodiments, the dosage will vary from about 1 mg to about 500 mg per kilogram of body weight of the mammal. In some embodiments, the dosage of the compound is about 500 mg per kilogram of body weight of the mammal. In some embodiments, the dosage of the compound is about 250 mg per kilogram of body weight of the mammal. In some embodiments, the dosage of the compound is about 200 mg per kilogram of body weight of the mammal. In some embodiments, the dosage of the compound is about 150 mg per kilogram of body weight of the mammal. In some embodiments, the dosage of the compound is about 100 mg per kilogram of body weight of the mammal. In some embodiments, the dosage of the compound is about 50 mg per kilogram of body weight of the mammal. In some embodiments, the dosage of the compound is about 25 mg per kilogram of body weight of the mammal. In some embodiments, the dosage of the compound is about 5 mg per kilogram of body weight of the mammal.

In some embodiments, the compound is administered in a fixed amount. In some embodiments, the dosage of the compound is between about 10 mg and about 10,000 mg. In some embodiments, the dosage of the compound is between about 100 mg and about 1,000 mg. In some embodiments, the dosage of the compound is between about 250 mg and about 750 mg. In some embodiments, the dosage of the compound is about 250 mg, about 300 mg, about 350 mg, about 400 mg, about 450 mg, about 500 mg, about 510 mg, about 520 mg, about 530 mg, about 540 mg, about 550 mg, about 560 mg, about 570 mg, about 580 mg, about 590 mg, about 600 mg, about 650 mg, about 700 mg, or about 750 mg.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In some embodiments, the compound is administered several times a day. In some embodiments, the compound is administered before, during, or after every meal. In some embodiments, the compound is administered before every meal. In some embodiments, the compound is administered during every meal. In some embodiments, the compound is administered after every meal. In some embodiments, the compound is administered on an empty stomach between meals.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Rhamnan Sulfate Reduces Atherosclerotic Plaque Formation and Vascular Inflammation

Many marine product-derived polysaccharides have a structure roughly analogous to the polysaccharides in the glycocalyx and have oral bioavailability (Laurienzo, P., et al., 2010, Marine Drugs, 8(9):2435-2465; Patil, N. P., et al., 2018, Frontiers in Cardiovascular Medicine, 5:153). Several of these compounds including laminarin sulfate and fucoidan have been explored as treatments for vascular disease and as anti-coagulants (Patil, N. P., et al., 2018, Frontiers in Cardiovascular Medicine, 5:153). Sulfated polysaccharides specifically have exhibited antioxidant and anti-inflammatory properties making them potential therapeutics for atherosclerosis (Patel, S., et al., 2012, 3 Biotech, 2(3):171-185). Rhamnan sulfate (RS) is a polysaccharide derived from green seaweeds including Monostroma nitidum. Rhamnan sulfate has a structure similar to endogenous glycosaminoglycans but is composed of a different backbone of polysaccharides which are primarily alpha-1,3-linked L-rhamnose residues that have sulfate groups. Thus, RS provides a rough approximation of the chemical structure of many of the glycans that compose the endogenous glycocalyx of the artery including that of heparan sulfate (Li, N., et al., 2017, Carbohydrate Polymers, 159:195-206). Rhamnan sulfate has been shown to decrease inflammation in vascular endothelial cells in vitro and inhibit hepatic lipogenesis in a zebrafish model (Okamoto, T., et al., 2019, Journal of Natural Medicines, 73(3):614-619; Zang, L., et al., 2015, Journal of Functional Foods, 17:364-370). Previous studies from our group indicate that RS has some anti-inflammatory properties and oral bioavailability. These properties make RS a promising candidate as an oral treatment for atherosclerosis.

Rhamnan Sulfate is Internalized by Macropinocytosis and is Dependent on Proliferation on vSMCs.

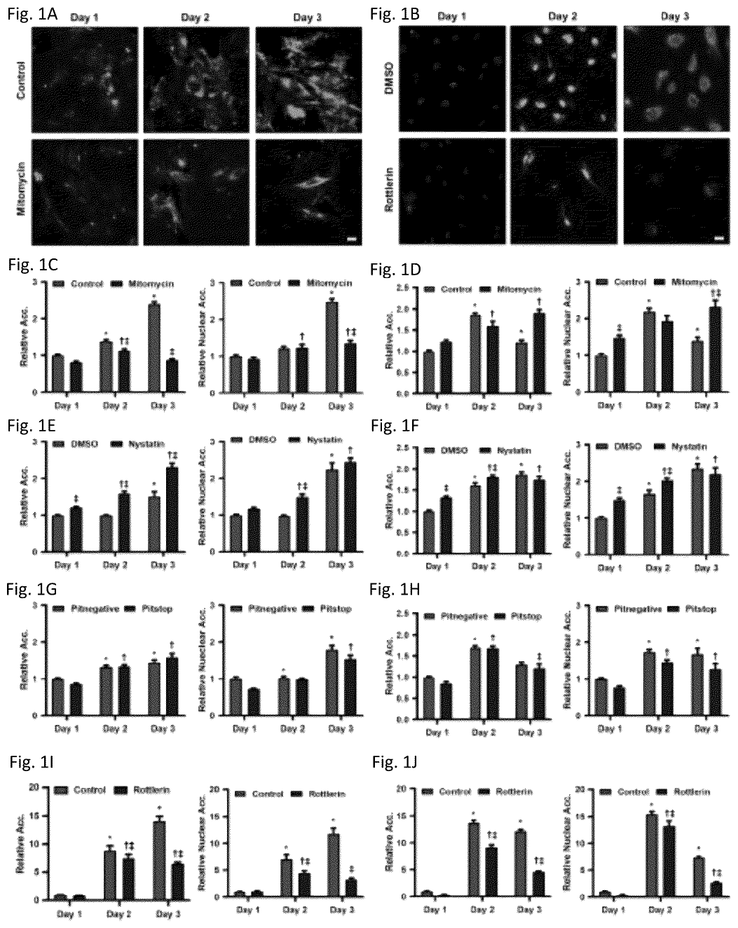

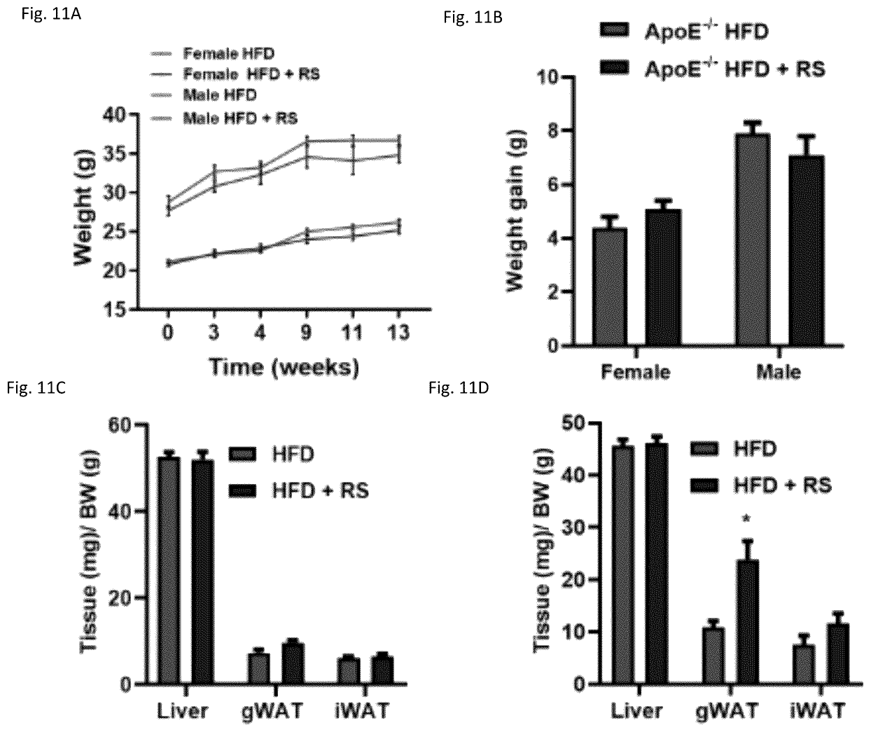

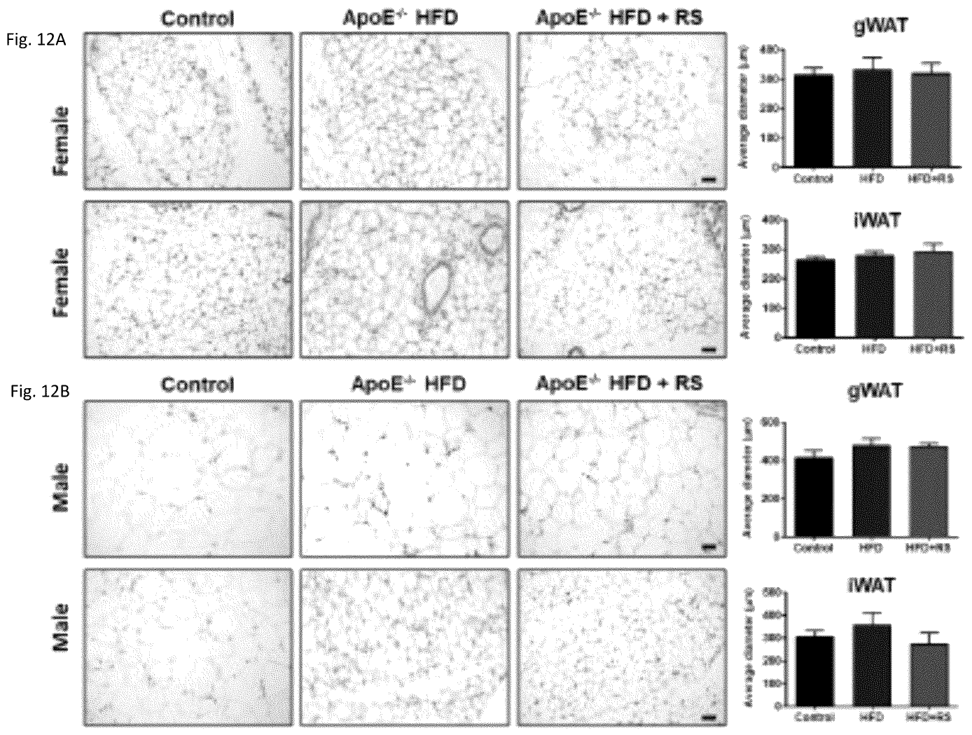

To examine the kinetics of uptake of RS by vascular cells, endothelial cells and aortic vSMCs were incubated with FITC-labeled RS. RS was detectable after 24 hours and there was nuclear fluorescence from the RS after 48 hours (FIGS. 1A and 1B). Based on the timing of the nuclear localization of RS, it was hypothesized that RS was entering the nucleus during the disassembly and reassembly of the nuclear envelope during cell division. To test this hypothesis, cells were mitotically arrested using mitomycin and RS uptake measured. In vSMCs, mitomycin significantly inhibited the uptake and nuclear localization of RS (FIG. 1C), however, this effect was not seen in endothelial cells (FIG. 1D). Cells were also treated with inhibitors of caveolin-mediated endocytosis (nystatin) and clathrin-mediated endocytosis (Pitstop® 2) but neither had clear inhibitory effects on RS uptake (FIGS. 1E-1H). In contrast, treatment with an inhibitor of macropinocytosis (rottlerin) significantly inhibited uptake of RS in both endothelial cells and vSMCs (FIGS. 11 and 1J).

Rhamnan Sulfate Decreases Proliferation and Migration of Vascular Cells.

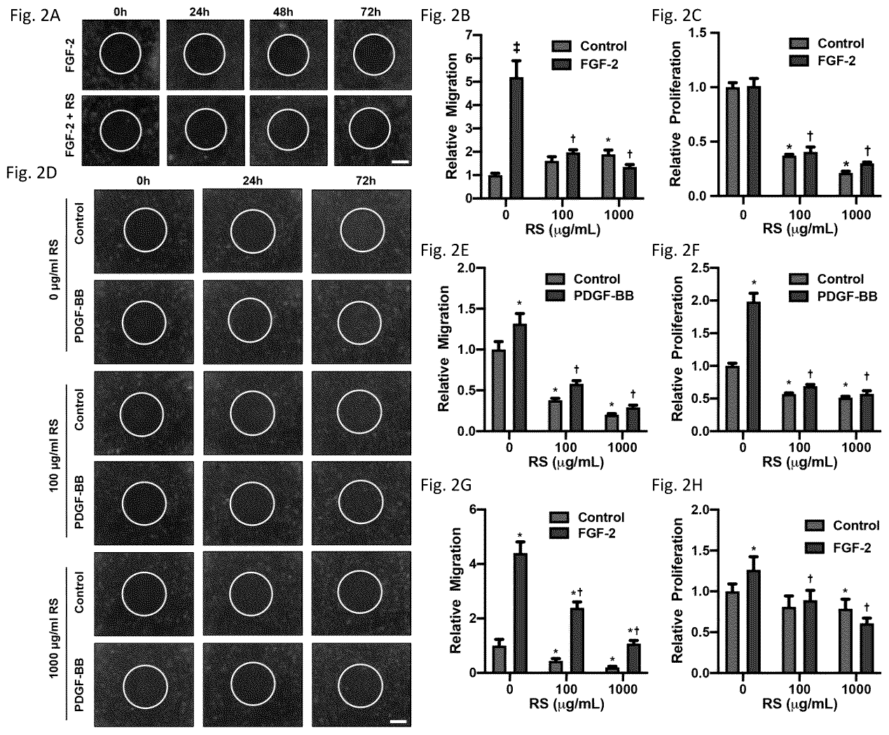

The proliferation and migration of vSMCs in atherosclerosis and vascular injury is an important mechanism in the development of intimal hyperplasia and remodeling of atherosclerotic plaques (Rudijanto, A., 2007, Acta Medica Indonesiana, 39(2):86-93). Endothelial proliferation and migration can be beneficial in vascular healing and reendothelialization following endothelial denudation (Lamalice, L., et al., 2007, Circulation Research, 100(6):782-794). However, these processes are also important for plaque neovascularization, which may have a role in thromboembolism and plaque destabilization (Gimbrone Jr., M. A., et al., 2016, Circulation Research, 118(4):620-636). Endothelial cells and vSMCs were treated with RS and their proliferation and migration assayed in response to growth factors. Treatment with RS significantly reduced proliferation and migration of endothelial cells treated with FGF-2 (FIGS. 2A-2C). In vSMCs, RS decreased proliferation and migration in response to PDGF-BB (FIGS. 2D-2F). It was also found that RS binds with high affinity to FGF-2 (KD=2.6×10⁻⁸ M) and PDGF-BB (KD=2.8×10⁻⁸ M; FIG. 3; Table 1). Proliferation and migration decreased in vSMCs treated with RS and FGF-2 (FIGS. 2G and 2H).

TABLE 1
Rhamnan sulfate binding affinities

| Interaction | kₐ (ms⁻¹) | k_{d} (s⁻¹) | K_{D} (M) |
|---|---|---|---|
| PDGF-BB to RS | 2.5 × 10⁵ ± 4.9 × 10³ | 5.4 × 10⁻³ ± 1.1 × 10⁻³ | 2.8 × 10⁻⁸ |
| PDGF-BB to Heparin | 2.6 × 10⁵ ± 4.3 × 10³ | 4.9 × 10⁻³ ± 8.3 × 10⁻⁵ | 1.9 × 10⁻⁸ |
| FGF-2 to RS | 3.0 × 10⁵ ± 6.0 × 10³ | 7.7 × 10⁻³ ± 8.1 × 10⁻⁵ | 2.6 × 10⁻⁸ |
| FGF-2 to Heparin | 5.9 × 10⁵ ± 1.4 × 10⁴ | 5.3 × 10⁻³ ± 8.1 × 10⁻⁵ | 9.0 × 10⁻⁹ |
| NFκB p50 to RS | 1.8 × 10⁴ ± 721 | 1.9 × 10⁻³ ± 7.9 × 10⁻⁵ | 1.1 × 10⁻⁷ |
| NFκB p50 to Heparain | 1.4 × 10⁴ ± 627 | 2.1 × 10⁻³ ± 8.4 × 10⁻⁵ | 1.5 × 10⁻⁷ |
| NFκB p65 to RS | 4.8 × 10⁵ ± 1.1 × 10⁴ | 0.011 ± 2.2 × 10⁻⁴ | 2.3 × 10⁻⁸ |
| NFκB p65 to Heparain | 2.6 × 10⁵ ± 5.2 × 10⁵ | 0.011 ± 3.4 × 10⁻⁴ | 4.2 × 10⁻⁸ |

Rhamnan Sulfate Enhances Endothelial Barrier Function.

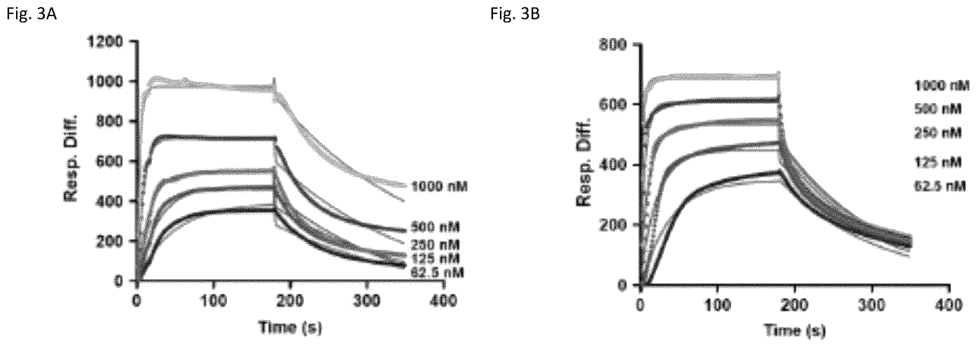

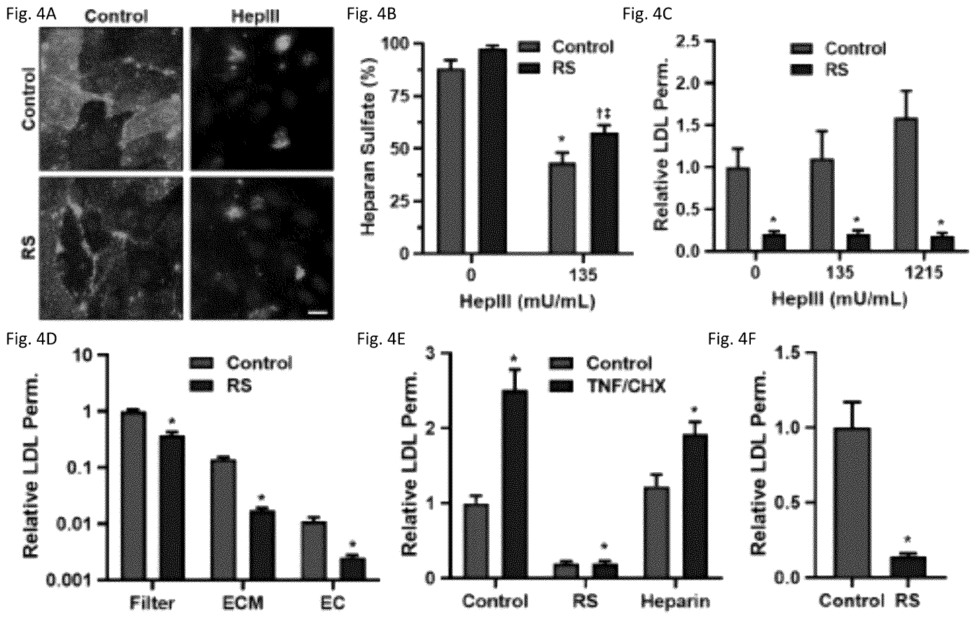

It was next examined whether RS could alter endothelial barrier function to LDL in the presence of treatments that induce inflammation or simulate the destruction of the glycocalyx. Endothelial cells were treated with heparinase III, a bacterial enzyme that degrades heparan sulfate. In untreated endothelial cells this led to a drop in heparan sulfate coverage of the cells from 96% to 36% of the cells (FIGS. 4A and 4B). With RS treatment, this heparinase-induced reduction in coverage was reduced to 59%, indicating that RS prevented the degradation of endogenous heparan sulfate glycosaminoglycans (FIGS. 4A and 4B). The permeability of the endothelium to LDL was lower in endothelial cells treated with RS, regardless of dose of heparinase applied (FIG. 4C). Using Transwell filters as the base, the effect of 30 minutes of RS incubation on the blank filter, subendothelial matrix, and endothelial monolayer was also tested. Treatment with RS reduced LDL permeability by 2.7-fold for the blank filter, 8-fold for the subendothelial matrix and 4.4-fold for the endothelial monolayer (FIG. 4D). These results indicated that RS accumulates both in the matrix and on the endothelial monolayer, where it can provide enhanced barrier function. The effectiveness of RS in reducing LDL permeability was also tested in endothelial cells after combined treatment with TNF-α and the protein synthesis inhibitor cycloheximide (CHX). While LDL permeability increased after the TNF-α/CHX treatment in control cells, it remained at basal levels with RS incubation (FIG. 4E). In contrast this effect was not observed with treatment with heparin (FIG. 4E). The permeability of endothelial monolayers to VLDL was also examined and it was found that it was lower with RS treatment (FIG. 4F). These results suggest that RS can reduce endothelial permeability to LDL and improve overall barrier function.

Rhamnan Sulfate Reduces TNF-α Induced NF-κB Activation in Human Endothelial Cells.

Atherosclerosis is a chronic disease characterized by inflammation, lipid accumulation and the progressive development of plaques (Stary, H. C., et al., 1994, Circulation, 89(5):2462-2478; Nielsen, L. B., et al., 1996, Atherosclerosis, 123(1-2):1-15). Lipid lowering therapies including statins have provided significant reductions in the risk of fatal coronary heart disease and non-fatal myocardial infarction. However, in spite of these advancements, atherosclerotic disease continues to be a pervasive clinical problem (Taylor, F., et al., 2013, The Cochrane Database of Systematic Review, 1:CD004816). Atherosclerosis slowly progresses for life, placing more strict requirements on pharmaceutical products to treat vascular disease in comparison to many other diseases. Compounds for treating atherosclerosis will need to be taken orally by patients on a daily basis for decades, requiring long-term safety and representing a major investment by healthcare systems to provide for patients.

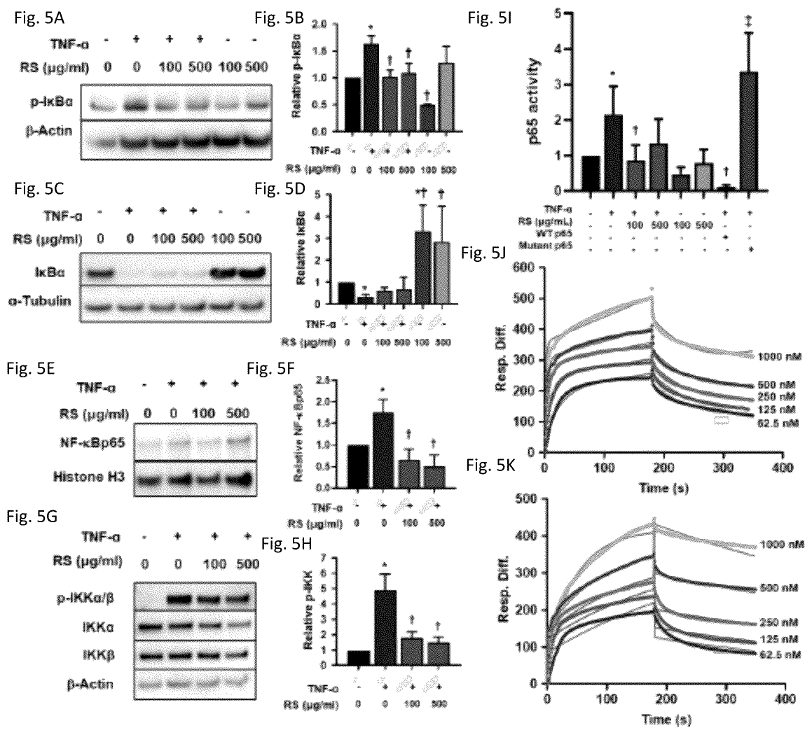

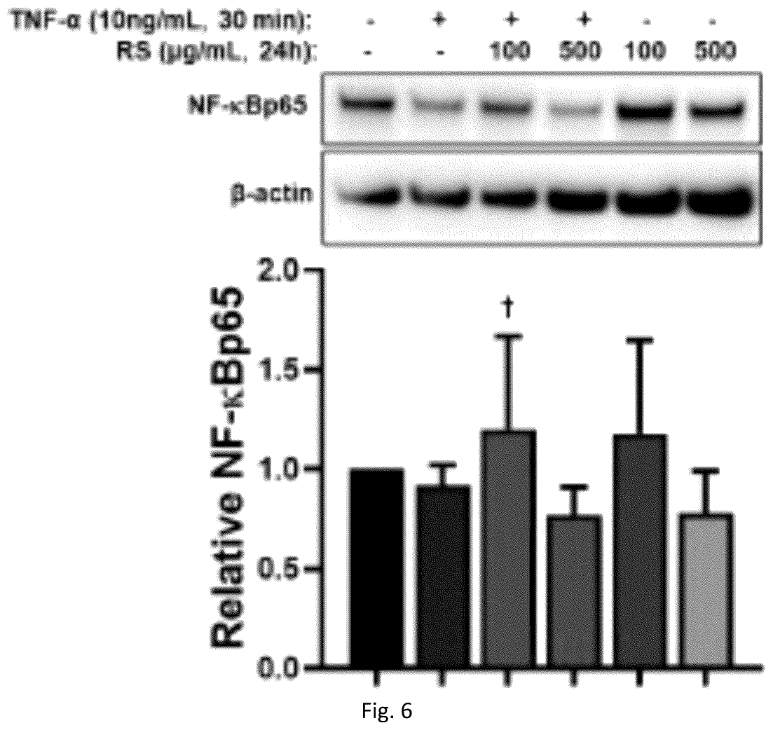

The NF-κB pathway is an important pathway in controlling endothelial inflammation and is implicated in atherosclerosis, lipid metabolism, and control of vSMC proliferation (Yu, X.-H., et al., 2015, Advances in Clinical Chemistry, 70(Chapter 1):1-30; Gareus, R., et al., 2008, Cell Metabolism 8(5):372-383). In the canonical NF-κB pathway, an NF-κB dimer is sequestered in the cytoplasm though interaction with a member of the IκB family of proteins (e.g., IκBα). Activation of a receptor leads to the recruitment of adaptor proteins followed by recruitment of the IκB kinase complex (IKK; typically consisting of IKKα, IKKβ and IKKγ), which subsequently phosphorylates IκB and eventually leads to its degradation. The destruction of IκB allows NF-κB dimers to translocate to the nucleus where they can control gene transcription. To examine if RS could alter signaling though the NF-κB pathway, endothelial cells were treated with TNF-α for varying times and the activation of NF-κB pathway signaling intermediates measured using Western blotting. TNF-α induced an increase in IκBα phosphorylation after 10 min of treatment, indicating activation of the NF-κB pathway and that treatment with RS reduced this response (FIGS. 5A and 5B). After 30 minutes of TNF-α treatment there was increased IκBα degradation and this was not affected by RS but basal levels of IκBα were increased (FIGS. 5C and 5D). NF-κBp65 protein levels were significantly decreased by RS treatment in the nuclear fraction and increased in the cytoplasm fraction after 30 minutes of TNF-α treatment, indicating reduced activation of the NF-κB pathway (FIGS. 5E, 5F, and 6). In addition, phosphorylation of IKK, the complex responsible for the phosphorylation of IκBα, was also significantly reduced by RS treatment (FIGS. 5G and 5H). Together, these findings indicate that RS affects multiple steps in the canonical pathway of NF-κB activation by TNF-α. To further confirm these findings, NF-κBp65 activity was directly measured using a TransAM assay and it was found that activity was reduced in the nuclear extracts of TNF-α treated endothelial cells (FIG. 5I). As nuclear localization of RS was observed in trafficking studies, it was hypothesized that RS binds directly to some of the components of the NF-κB pathway. Using surface plasmon resonance, it was found that RS binds with high affinity to NF-κBp50 (KD=1.1×10⁻⁷ M) and NF-κBp65 (KD=2.3×10⁻⁸ M; FIGS. 5J and 5K). The dissociation constant for the proteins for binding to RS was comparable to binding with heparin (Table 1).

Rhamnan Sulfate has Oral Bioavailability and is Found in Vascular Tissues after Oral Administration.

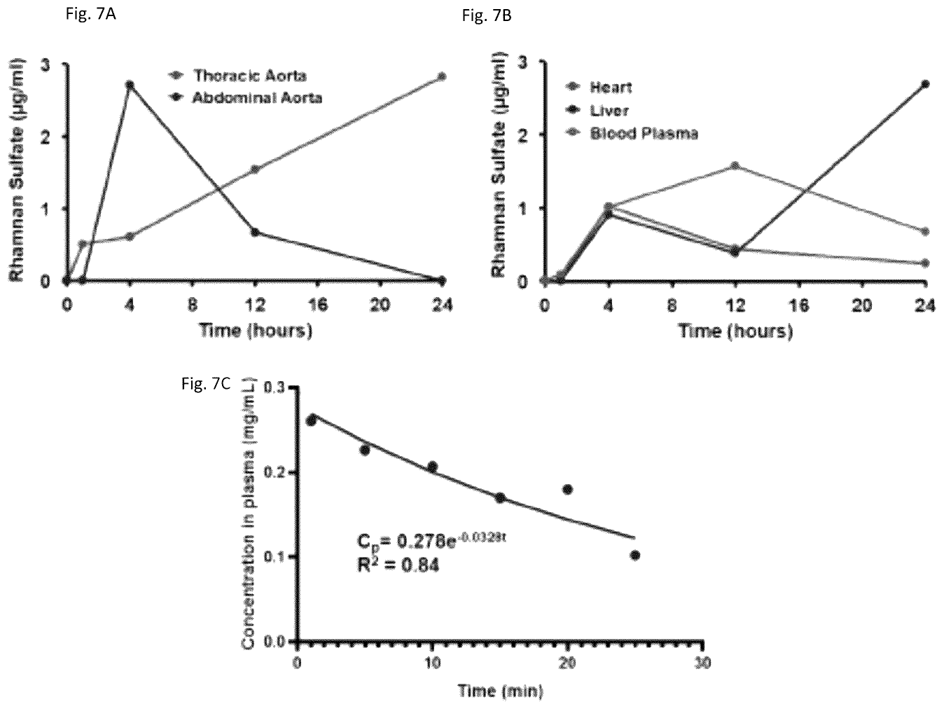

Prior to conducting an in vivo experiment with oral RS, the pharmacokinetics of the drug were studied to calculate the clearance rate and uptake by the aorta, heart, blood, and liver. RS was introduced to mice though oral gavage and the concentration in tissues measured over 24 hours. RS concentration started decreasing in the abdominal aorta after 4 hours but continued to rise steadily in the thoracic aorta for 24 hours (FIG. 7A). In the heart and total blood plasma, RS concentrations increased up to 4 and 12 hours respectively before being cleared (FIG. 7B). In the liver, there was an initial influx of RS at 4 hours and then a gradual increase in concentration after 12 hours (FIG. 7B). FITC labeled RS was also injected into the blood directly and its clearance examined. The half-life of RS is approximately 21.1 minutes in the blood following intravenous injection (FIG. 7C).

Orally Administered Rhamnan Sulfate Decreases Plaque Deposition in ApoE−/− Mice on a High Fat Diet.

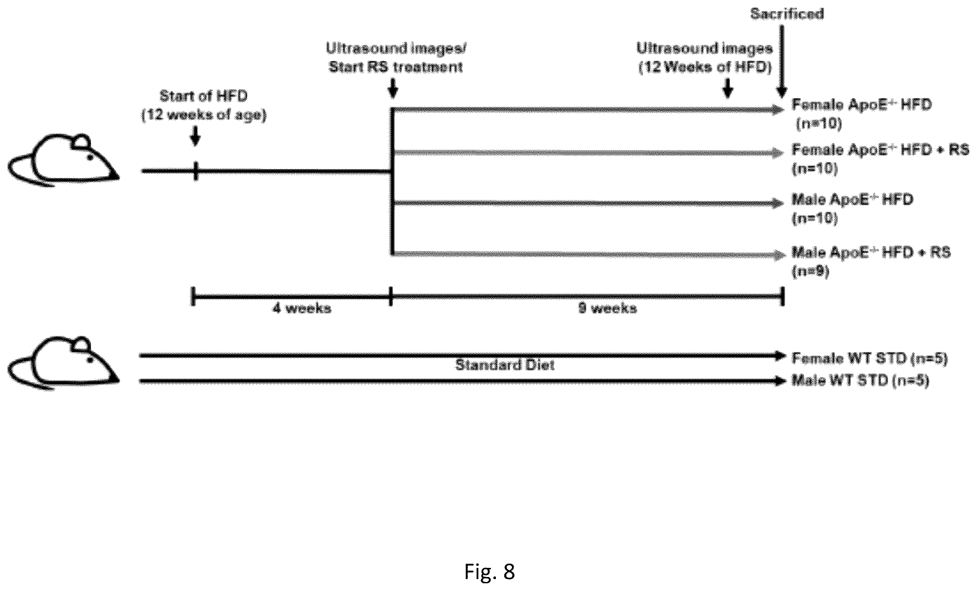

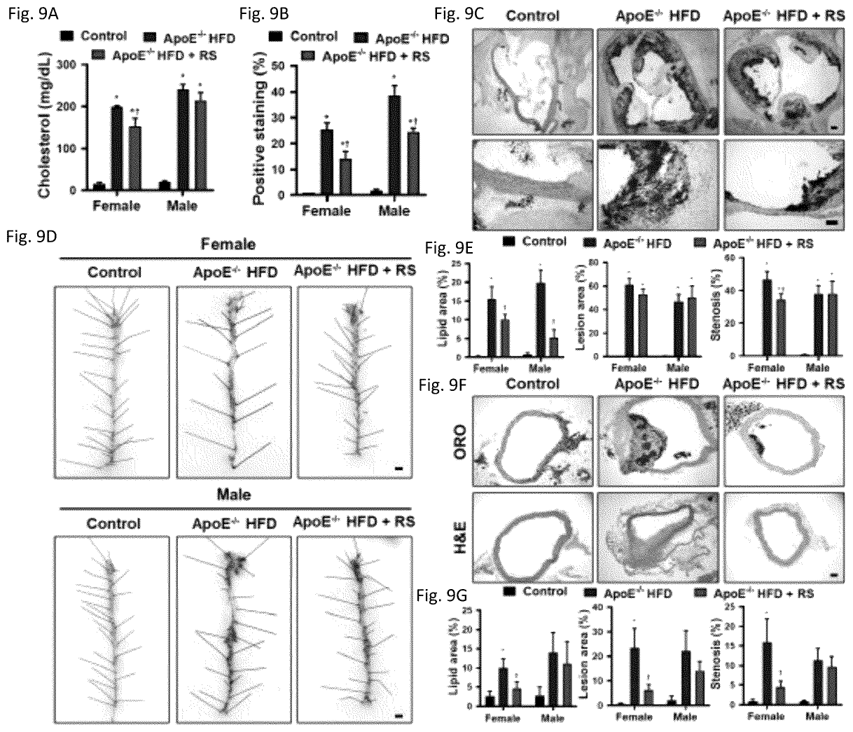

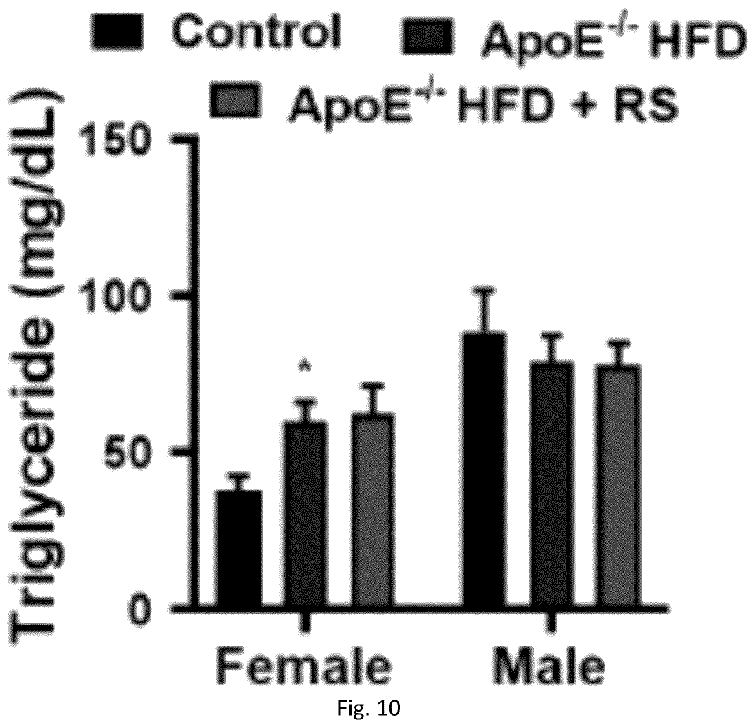

To test the effect of RS on the progression of atherosclerosis, ApoE−/− mice were fed a high fat diet or a high fat diet supplemented with RS for 13 weeks (FIG. 8). Blood cholesterol levels were decreased significantly in female ApoE−/− mice treated with RS relative to untreated mice but this was not observed in male mice (FIG. 9A). There was no change in plasma triglyceride levels in both male and female mice (FIG. 10). In en face preparations of the aorta, lipid deposition was decreased by 45.2% in female and 36.4% in male mice whole aortas with RS treatment in comparison to the ApoE^{-/-} HFD group (FIGS. 9B and 9C). In the aortic arch there was also significant decrease in lipid deposition in the male and female ApoE−/− mice with RS treatment. Stenosis decreased in female mice only (FIGS. 9D and 9E). In the thoracic aorta there was a reduction in lipid deposition, lesion area, and stenosis for female mice treated with RS but not for male mice (FIGS. 9F and 9G).

Oral Rhamnan Sulfate does not Affect Body Weight or Adipose Tissue Deposition in ApoE−/− Mice.

There was no difference in the weight of the mice between the RS treated and untreated mice for both male and female groups (FIGS. 11A and 11B). There was also no significant difference in the ratio of liver, inguinal white adipose tissue (iWAT), and gonadal white adipose tissue (gWAT) to total body weight in female mice (FIG. 11C). Characterization of the iWAT and gWAT also showed no change in size of adipocytes in treated female mice (FIG. 12A). In male mice, the ratio of gWAT to total body weight increased in mice treated with RS (FIG. 11D). The size of adipocytes remained the same in both iWAT and gWAT in the HFD and HFD+RS treated groups (FIG. 12B).

Rhamnan Sulfate Treatment Reduces High Fat Diet-Induced Increases in Blood Velocity in ApoE−/− Mice.

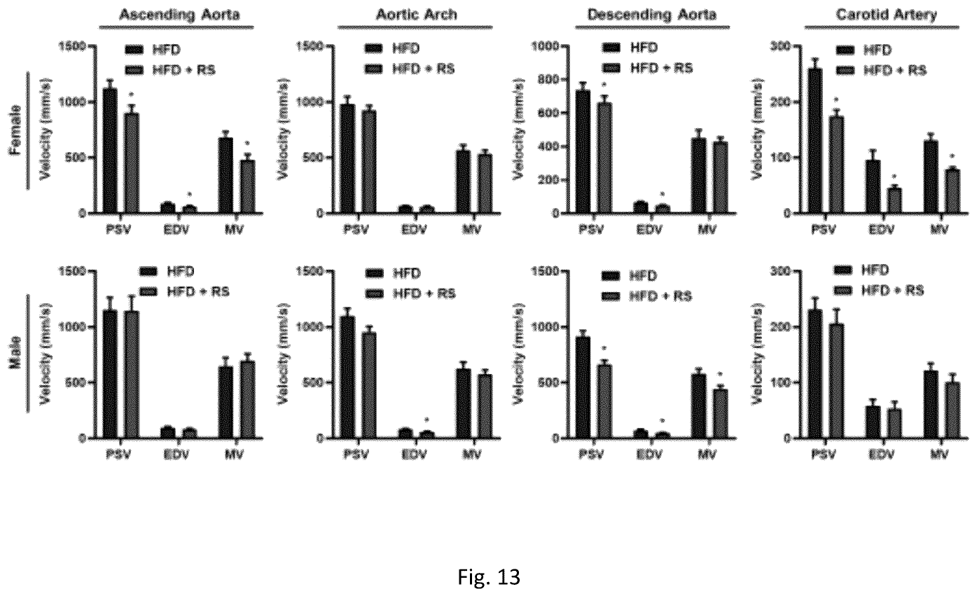

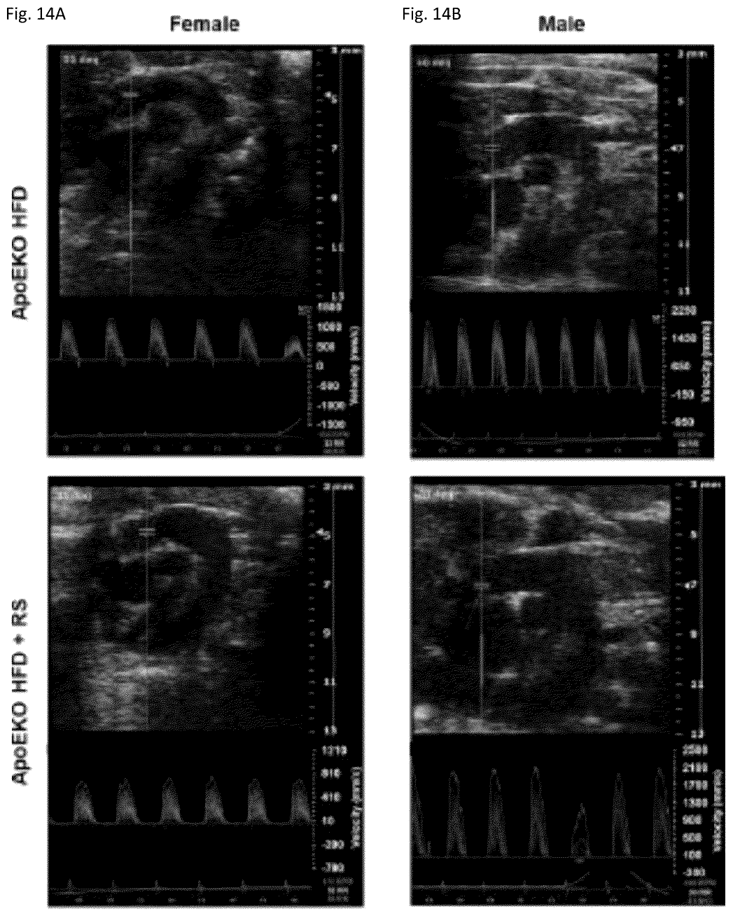

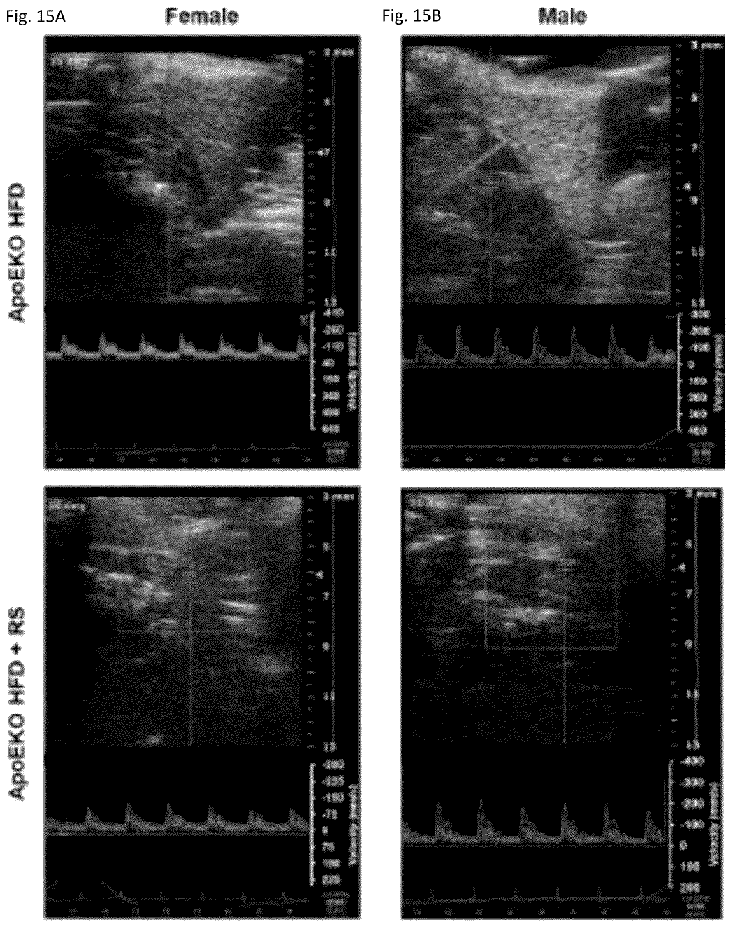

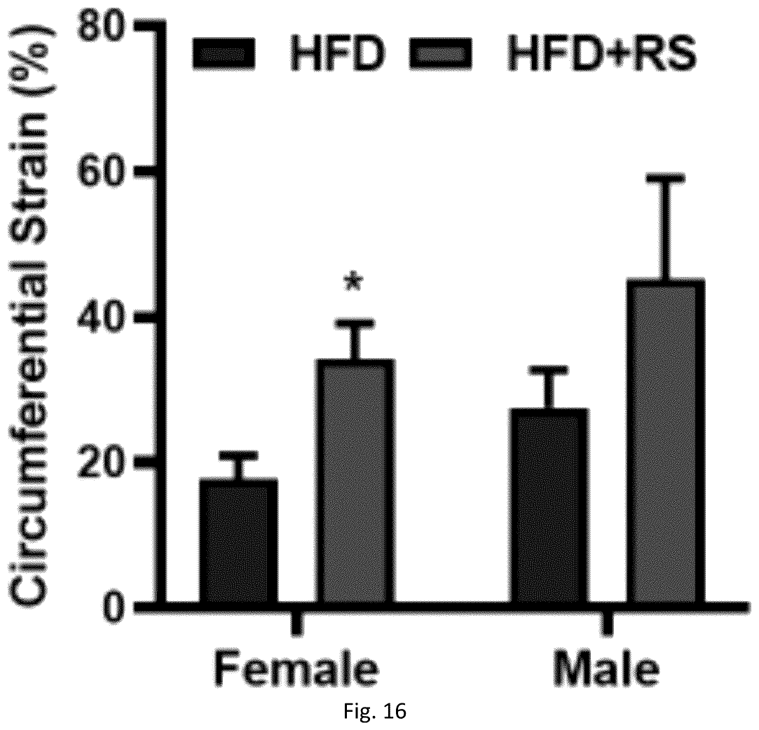

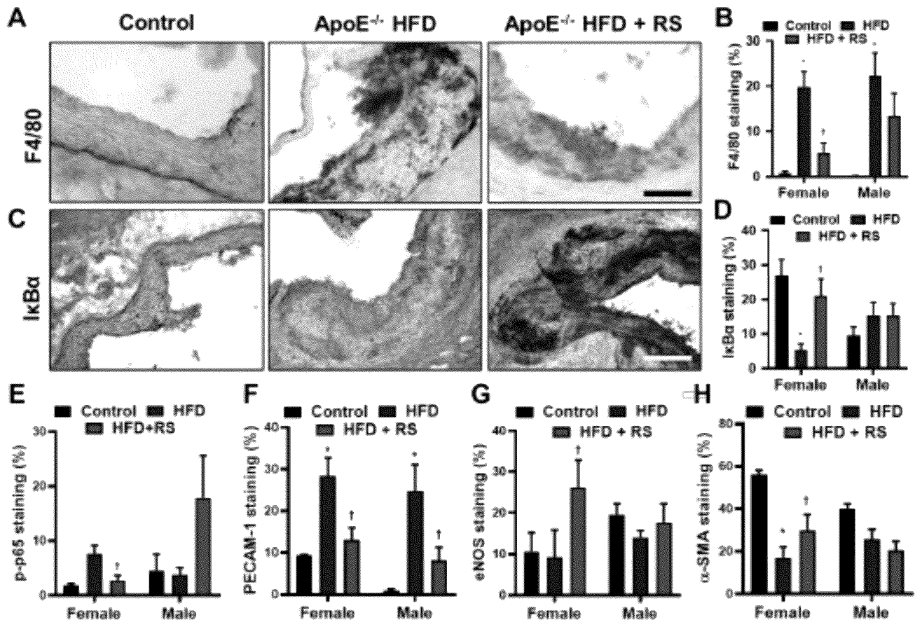

To monitor plaque development over the course of the experiment, blood velocity was measured in the aorta and left common carotid arteries of ApoE−/− mice. Peak systolic velocity (PSV), end diastolic velocity (EDV), and mean velocity (MV) were calculated in the ascending and descending aorta, aortic arch, and carotid artery. In female mice, PSV, EDV, and MV decreased with RS treatment in the ascending aorta (FIGS. 13 and 14A). In the aortic arch for female mice, there was no change in blood flow velocities with RS treatment but there was a significant decrease in flow velocities in the descending aorta for the PSV and EDV with RS treatment (FIG. 13). All flow velocities decreased in the carotid artery with RS treatment as well for female mice (FIGS. 13 and 15A). In male mice, there was no change in all three velocities in the ascending aorta and only EDV decreased in the aortic arch (FIGS. 13 and 14B). In the descending aorta, PSV, EDV, and MV all decreased with RS treatment while there was no change in the velocities in the carotid artery (FIGS. 13 and 15B). In addition, it was found that the elasticity, measured by circumferential strain, of the aorta during the cardiac cycle was higher in female mice treated with RS (FIG. 16).

Rhamnan Sulfate Reduces Vascular Inflammation in Female Mice but not Male Mice.

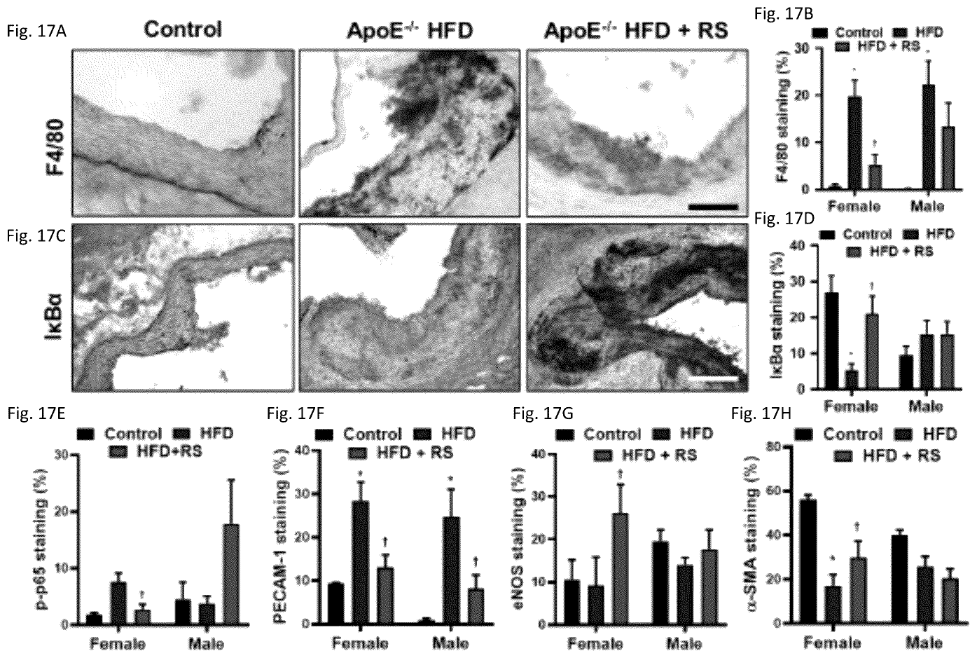

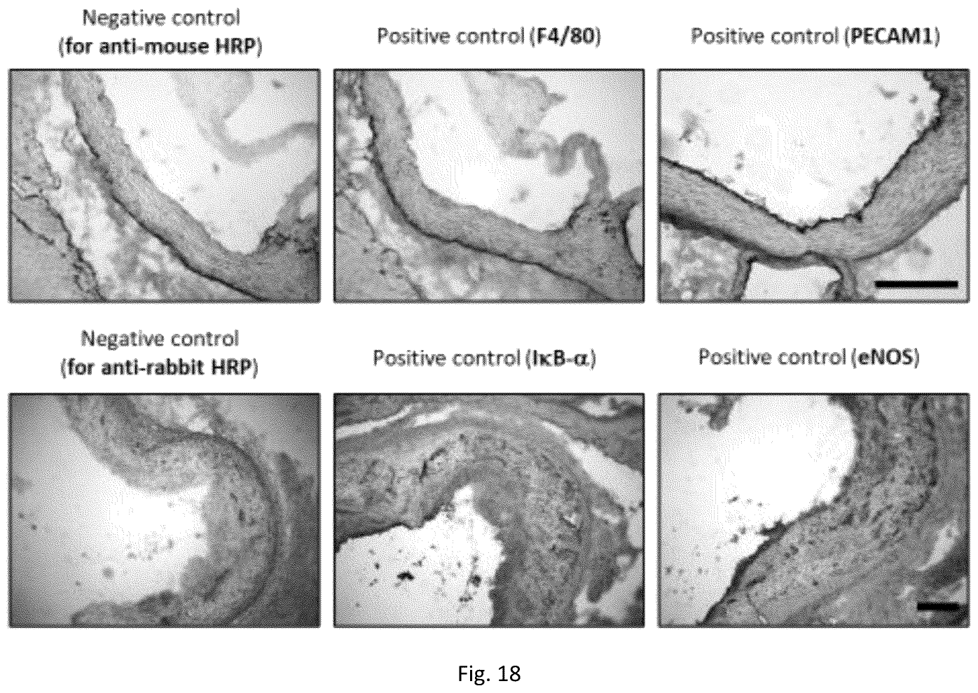

To quantify the efficacy of oral RS in reducing vascular inflammation, the presence of macrophages and activation of the NF-κB pathway were examined in the ApoE−/− mice treated with HFD or HFD with RS supplementation. There was a significant decrease in macrophages (F4/80 positive cells) in histological sections from the aortic root in female mice treated with RS in comparison to the HFD group but there was no change in male mice (FIGS. 17A and 17B). Also, IκBα, an inhibitor of NF-κB, was significantly upregulated in female mice (FIGS. 17C and 17D). Phosphorylation of the p65 subunit of NF-κB was also reduced in female ApoE−/− mice on a HFD treated with RS (FIGS. 17E and 18). Phosphorylation of p65, IκBα and F4/80 were not affected by RS treatment in male mice (FIGS. 17B, 17D, and 17E).

Treatment with RS Reduces Plaque Angiogenesis and Enhances Endothelial eNOS Production in ApoE−/− Mice on a High Fat Diet.

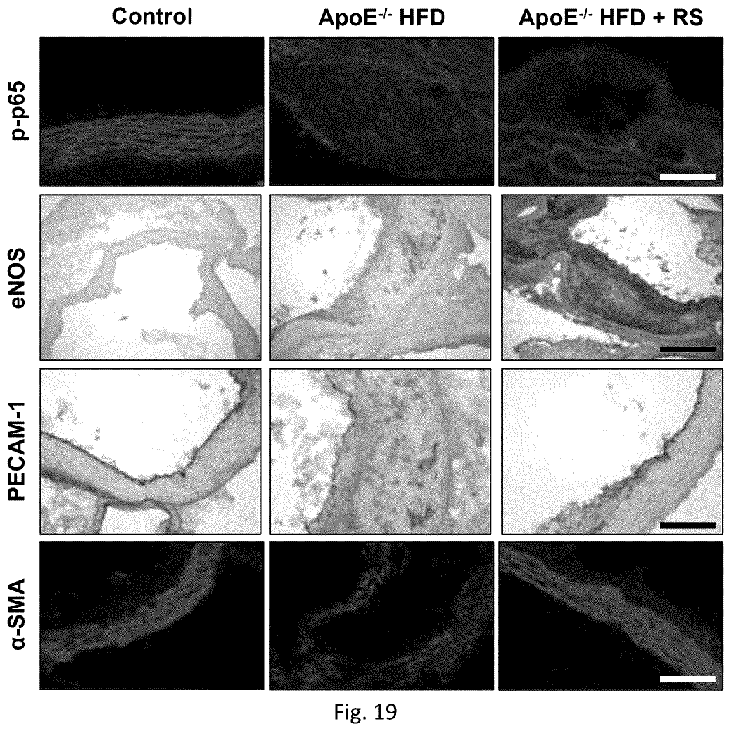

During atherosclerotic inflammation, endothelial permeability is increased by PECAM-1 upregulation as it promotes leukocyte transmigration and integrin activation. In both female and male ApoE−/− mice, PECAM-1 was decreased with RS treatment (FIGS. 17F and 19). Impaired eNOS levels lead to atherogenesis though increased nitric oxide breakdown and superoxide production (Kawashima, S., et al., 2004, Arteriosclerosis, Thrombosis, and Vascular Biology, 24(6):998-1005). In female mice treated with RS, there was higher eNOS production in the aortic arch (FIGS. 17G and 19). There was an increased area of α-SMA staining in the aortic arch of female ApoE−/− mice treated with RS, reflecting the reduction in plaque size (FIGS. 17H and 19). In male mice, eNOS and α-SMA were not affected by RS treatment (FIGS. 17G and 17H).

These studies demonstrate that rhamnan sulfate has potent anti-inflammatory properties, reduces vSMC proliferation, and reduces atherosclerosis in a hyperlipidemic mouse model. As rhamnan sulfate is relatively inexpensive and has been consumed by millions of people as part of their diet, it would present an easily implementable adjuvant therapy to lipid lowering drugs and other treatments for atherosclerotic disease if it were effective in human patients.

Rhamnan sulfate has atheroprotective effects though multiple modes of action. It reduces migration of vascular cells and binds to pro-growth and inflammatory growth factors with an affinity similar to that of heparin, suppressing the growth of vSMCs. This effect proceeds through binding of growth factors including FGF-2 and PDGF-BB. However, RS also inhibits migration in the absence of stimulation with these factors suggesting it also acts by directly altering cellular adhesion/migration mechanisms. The uptake of RS was reduced in both endothelial and vascular smooth muscle cells with rottlerin treatment, indicating that a major mechanism of entry into the cells is macropinocytosis. In vSMCs, mitomycin treatment also reduced nuclear levels of RS, implying that RS may enter the nucleus during mitosis. This effect may be more pronounced in vSMCs due to their higher proliferation rate.

Rhamnan sulfate accumulates in vascular cells over the course of several days and is also deposited in the extracellular matrix. Interestingly, rhamnan sulfate appears to be able to access and bind to nuclear protein during cell division. This finding suggests a novel mechanism of action for RS, in which it accumulates in the cytoplasm of the cells followed by binding to nuclear proteins, including NF-κB, during cell division. The polyanionic structure of rhamnan sulfate may mimic that of DNA, allowing it to competitively bind to regions of proteins that bind to DNA. In comparison, heparin can facilitate the nuclear entry of growth factors but heparin and its anti-proliferative derivatives do not enter the nucleus of vSMCs (Barxu, T., et al., 1996, Journal of Cell Physiology, 167(1):8-21). Prior studies have also suggested that the cellular localization of low molecular weight heparin is dependent on sulfation; however, none of the modified forms are found in the nucleus (Raman, K., et al., 2013, Molecular Pharmaceutics, 10(4):1442-1449). Thus, rhamnan sulfate is not simply acting as a heparin analogue in this activity.

A striking feature is the differences in response between male and female animals treated with rhamnan sulfate. In the blood vessels of mice treated with rhamnan sulfate, at the aortic root there was a more pronounced reduction in lipid area for male mice, but the overall lesion and stenotic response was only significantly lower in females. In the thoracic aorta, only female mice had a reduction in plaque size and stenosis. A stronger cholesterol lowering effect of rhamnan sulfate was also observed in female mice compared to male mice. Lipid lowering effects have been observed for marine polysaccharides including fucoidan, ulvan, and laminarin sulfate (Zaporozhets, T., et al., 2016, Pharmaceutical Biology, 54(12):3126-3135; Hassan, S., et al., 2011, Saudi Journal of Biological Sciences, 18(4):333-340; Besterman, E., et al., 1970, Atherosclerosis, 12(1):85-96). In comparison to these, rhamnan sulfate did not produce as dramatic reductions in lipids. The anti-inflammatory properties of rhamnan sulfate appeared to be stronger in female mice as well, including significant lowering of plaque inflammation and lipids with rhamnan sulfate treatment.

Overall, this demonstrates that rhamnan sulfate can provide significant benefits for inhibiting the development of atherosclerosis and acts though multiple mechanisms including the reduction of inflammation, inhibition of vascular cell proliferation and enhanced endothelial barrier function. Due to the low cost and ease of availability, RS may have high potential as an easily implementable adjunct therapy for atherosclerosis. These studies used a highly purified form of rhamnan sulfate, which may allow it to have increased binding capacity and activity in comparison to less pure forms. Further studies are ongoing to verify that less purified forms of the compound have similar activity or if particular subfractions of RS can be linked to specific mechanistic activities.

### Example 2: Altered Gene Expression Resulting from Rhamnan Sulfate Treatment

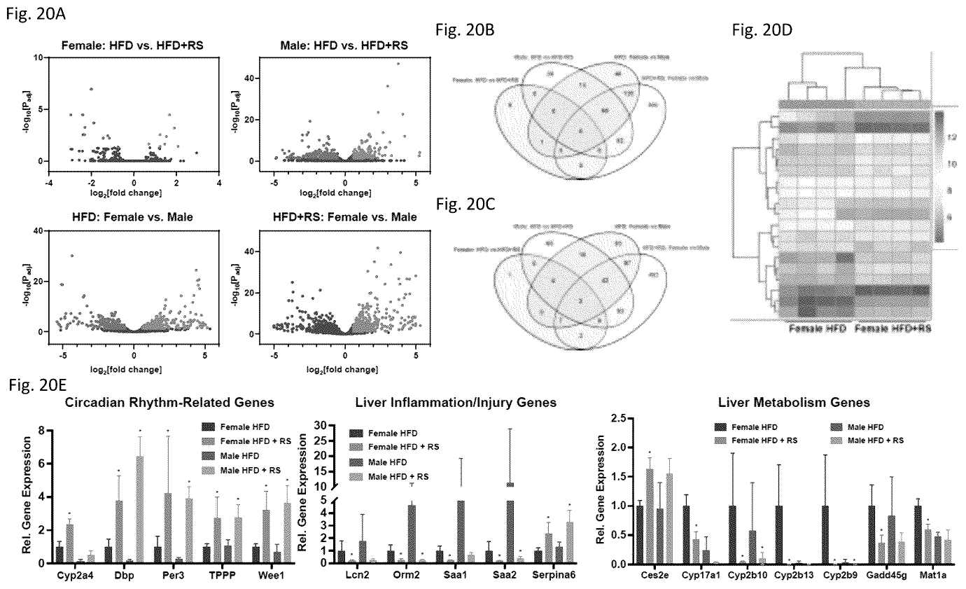

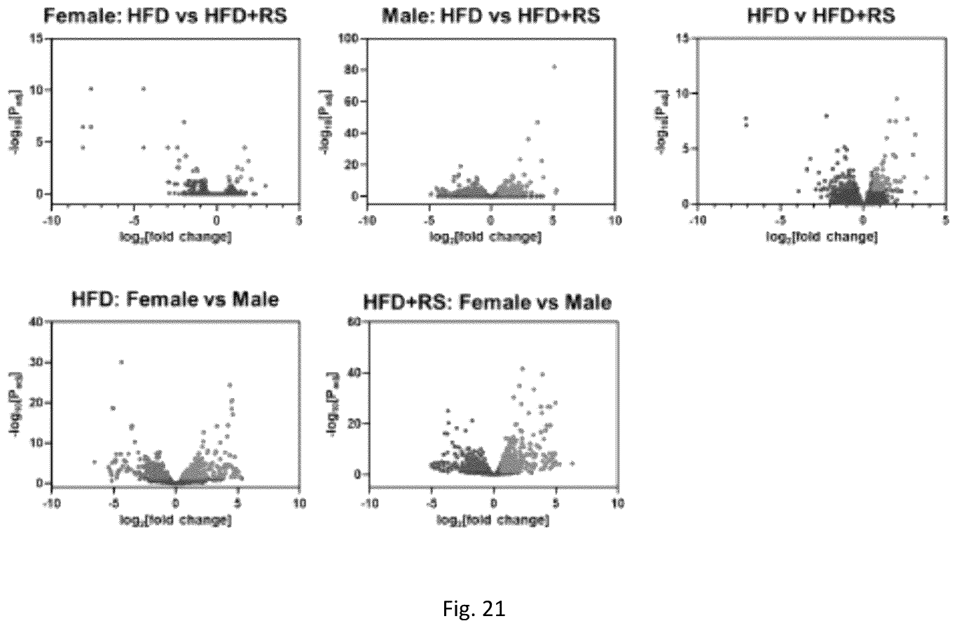

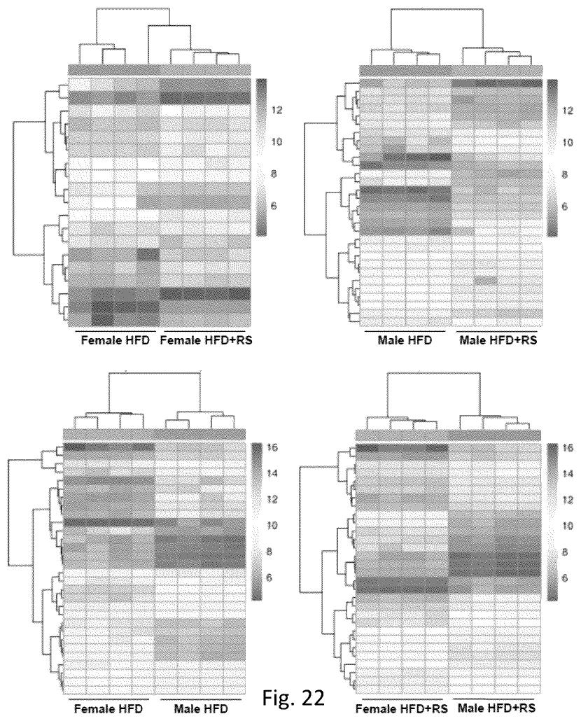

To understand the sex-specific differences in the response to rhamnan sulfate (RS), a transcriptomic analysis was performed on the livers of ApoE mice used in Example 1. A differential gene expression analysis revealed that RS treatment only a limited number of genes were significantly altered by RS in female mice but a much larger number of genes was altered by RS in male mice (FIGS. 20A, 20B, and 21). While a few genes were commonly altered by RS treatment in both male and female mice, there were a large set of differentially regulated genes between the sexes (FIGS. 20B-20D and 22). A gene ontology analysis showed that in male mice treated with RS there were significant changes in circadian rhythm an lipid metabolism pathways (FIG. 23A), while a similar analysis in female mice demonstrated changes in lipid and drug metabolism pathways (FIG. 23B). An analysis of individual genes thar were commonly regulated between the male and female mice showed significant alteration in circadian rhythm-related and -regulated genes, liver inflammation or injury genes, and liver metabolism genes (FIG. 20E). Specifically, an upregulation of the circadian rhythm-related gene PER3, a major regulator of circadian rhythm in peripheral tissues, was observed (Archer, S. N., et al., 2018, Sleep Medicine Reviews, 40:109-126). In addition, there was a significant down regulation in several genes relating to liver inflammation. Major shifts in liber metabolism-related genes were also observed in female mice.

### Example 3: Hepatic Steatosis is Decreased by Oral Rhamnan Sulfate Treatment

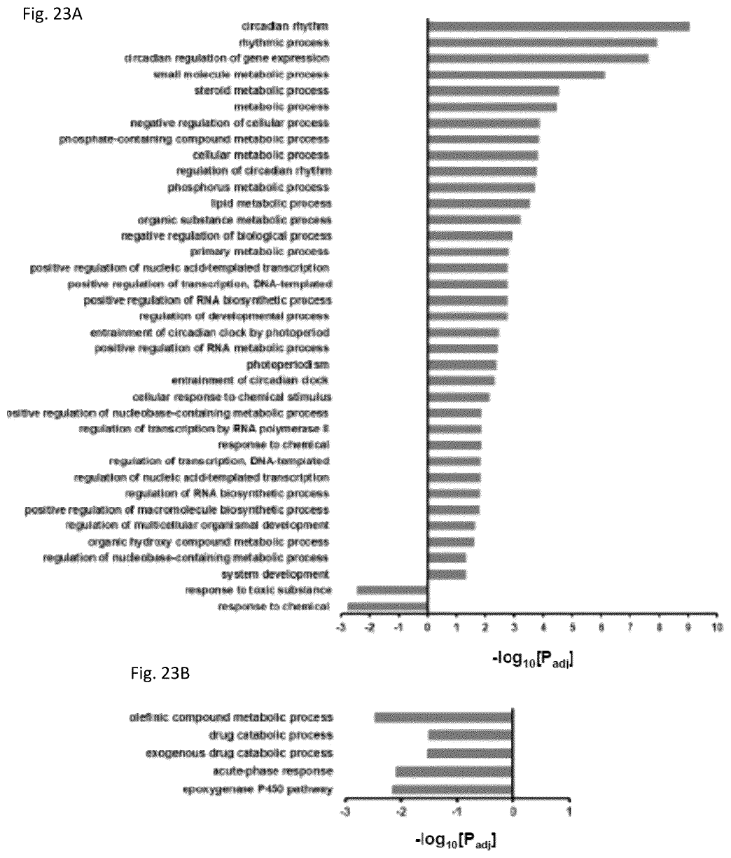

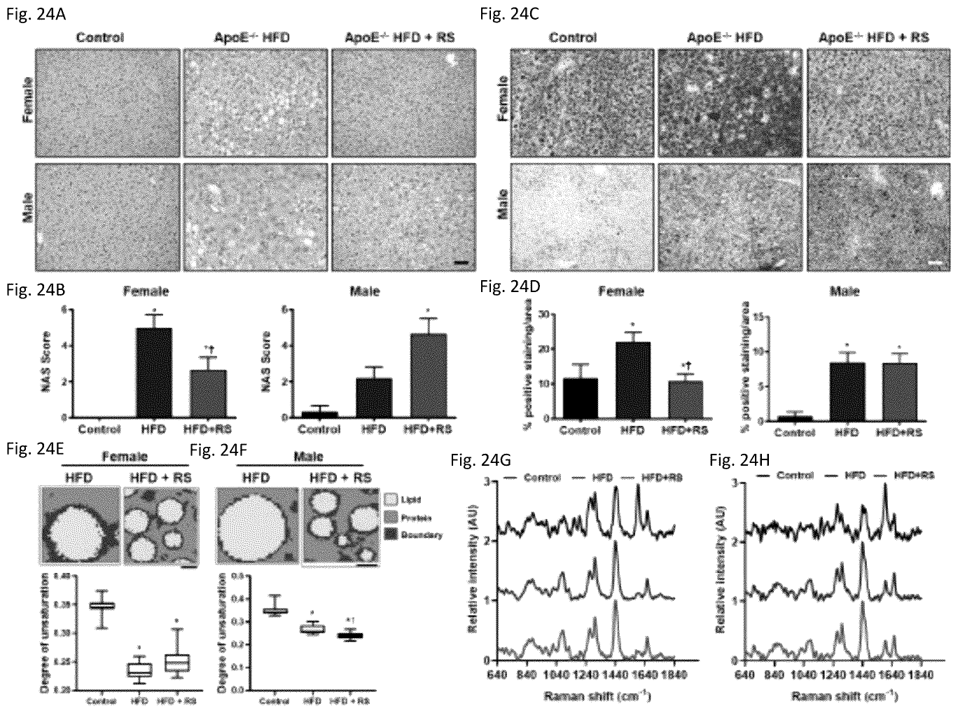

Non-alcoholic fatty liver disease (NAFLD) has been correlated with the development of atherosclerosis and is linked to inflammation, metabolic syndrome and obesity (Zhou, Y. Y., et al., 2018, Hepatology Communications, 2(4):376-392; Muhammad, R. S., et al., 2018, Atherosclerosis, 276:155-162). To test the effect of RS on hepatic lipid deposition, histological analysis and Raman spectroscopy were performed on tissue sections from ApoE−/− mice. NAS scores (a sum of histopathological scores for steatosis, inflammation and ballooning) were quantified for the livers from the mice treated with HFD or HFD+RS. In female mice, NAS scores were reduced with RS treatment but appeared increased in male mice (FIGS. 24A and 24B). There was also a marked reduction in lipid deposition in the livers of female mice but no change in lipid deposition for male mice treated with RS (FIGS. 24C and 24D). Analysis of the livers with Raman spectroscopy indicated that the lipids in male mice livers were less saturated in the RS treated group in comparison to the HFD group (FIGS. 24E-24G). In addition, the overall size of lipid droplets decreased in both male and female livers after RS treatment (FIG. 24E).

The methods employed in all Examples are described herein.

Cell culture. Human umbilical vein endothelial cells (HUVECs) were purchased from PromoCell. They were grown in MCDB-131 culture medium (Life Technologies) with EGM-2 growth factors (R&D Systems), 10% fetal bovine serum (FBS), L-glutamine and antibiotics. Human aortic smooth muscle cells (HAoSMCs) were grown in MCDB-131 culture medium (Life Technologies) with 10% FBS, L-glutamine and antibiotics. Cells were received at passage 2 and were not allowed to grow past passage 8. Human coronary artery endothelial cells (HCAEC) were purchased and grown in cell specific growth medium according to the manufacturer's instructions (Cell Applications). All cells were grown at 37° C. with 5% CO₂.

Rhamnan sulfate purification/analysis. Rhamnan sulfate was isolated from green seaweed (Monostroma nitidium sourced from Japan) powder using methods described previously (Liu, X., et al., 2018, Marine Drugs, 16(7):243). The powder was homogenized in distilled water (1:30 seaweed:water) and extraction was performed at 100° C. for three hours. The extract was centrifuged at 4700 g for 10 min and the supernatant was collected. Ethanol was added to the supernatant to achieve 80% ethanol per total volume. Crude polysaccharide was precipitated from the supernatant with three volumes of anhydrous ethanol and dissolved in distilled water. The polysaccharide solution was dialyzed in a cellulose membrane (molecular weight cut-off of 3500 Da) against distilled water for three successive days. After dialysis, the RS was lyophilized and weighed. Molecular weight of a monomer of RS was 150 kDa, measured after purification, and the aggregate in 1 mg/mL solution was estimated to be 2.67×10⁷ kDa using DLS.

Labeling of RS with FITC. Labeling and detection of RS with FITC was performed as described previously (Glabe, C. G., et al., 1983, Analytical Biochemistry, 130(2):287-294). Briefly, the polysaccharide was activated by adding CNBr (8.33 mg/mL) to RS (20 mg/mL) with the mixture maintained at pH 11. Activated RS was desalted using a 20 cm Sephadex G-50 column in 0.2 M sodium borate (pH 8.0). The RS-containing fractions were pooled and reacted with 2 mg fluoresceinamine. Gel filtration was used to separate RS-FITC from unreacted fluoresceinamine and the concentration of fluorescein determined by reading absorbance at 440 nm. Concentration of RS was determined using the phenol-sulfuric acid method. Degree of addition was expressed as moles of fluorescein per mole of monosaccharide and molecules of fluorescein per polysaccharide molecule.

Inhibition of uptake pathways. Endothelial and vascular smooth muscle cells were grown to confluence in MCDB-131 medium as described earlier. To prevent mitosis, mitomycin (1 mg/mL; Sigma-Aldrich) was added to the cells, with no treatment cells as control. To prevent uptake through the caveolin-mediated endocytosis pathway, nystatin (1 mg/mL; Thermo Fisher Scientific) was added to the cells, using DMSO (1 mg/mL) for control. Pitstop® 2 and Pitstop® 2-negative control (1 mg/mL; Abcam) were added to both cell types to prevent uptake through the clathrin mediated endocytosis pathway. Rottlerin (1 mg/mL; Santa Cruz Biotechnology) and DMSO control were used to test for inhibition of macropinocytosis. All groups received 1 mg/mL RS labeled with FITC. Cells were fixed after treatments for 24, 48, and 72 h, stained with DAPI, and imaged using a confocal microscope.

Proliferation and migration assays. To measure proliferation, cells were seeded on a fibronectin-coated 96-well plate and grown to confluence. Cells were starved with 0.5% FBS medium for 24 hours, then treated with RS (100 or 100011 g/mL) and growth factors (10 ng/mL PDGF-BB or FGF-2) for 72 hours. Proliferation was measured using the BrdU Cell Proliferation Assay Kit (Cell Signaling Technology). Migration was measured using the ORIS Cell Migration Assay Kit (Platypus Technologies). Cells were seeded on a fibronectin-coated ORIS plate and grown to confluence. The cells were then placed in media with 0.5% FBS for 24 hours. The stoppers were removed, and the cells were treated with RS (100 or 100011 g/mL) and growth factors (10 ng/mL PDGF-BB or FGF-2). Images of each well were captured every 24 hours for the duration of the experiment using a Cytation™ 5 Cell Imaging Multi-Mode Reader (BioTek).

LDL permeability assay. Human coronary artery endothelial cells (HCAECs) at passages 4-8 were plated onto fibronectin-coated Transwell membranes (12 mm diameter, 0.4 μm pores, Corning) at a density of 0.5×10⁴ cells/cm². Cells reached confluence within 2-3 days after plating, and experiments were carried out on monolayers 4-6 days post-plating. Immediately before the start of an experiment the media was changed to the experimental media consisting of phenol red-free basal media (Cell Applications) supplemented with 1% bovine serum albumin (BSA; Sigma-Aldrich). For studies with treatments, the HCAECs were incubated with growth media (control) or growth media containing RS at 25 μg/mL for 24 h, followed by a 2 h incubation with heparinase III (HepIII; Ibex pharmaceuticals, Quebec, Canada) at 135 mU/mL or 1215 mU/mL in experimental media. After the 2 h HepIII treatment, HCAECs were rinsed twice with experimental media and the permeability of DiI-LDL was measured. Tumor necrosis factor alpha (TNF-α, 20 ng/mL; Sigma-Aldrich) and cycloheximide (CHX, 3 mg/mL; Sigma-Aldrich) were used to induce elevated apoptosis and permeability (Cancel, L. M., et al., 2010, Atherosclerosis, 208(2):335-341). The cells were grown for 5 days before incubation with TNF-α and CHX (TNF-α/CHX) in the presence or absence of the RS isoforms or heparin (100 μg/mL; Sigma-Aldrich). TNF-α/CHX was removed after 3.5 h of incubation and the cells were allowed to recover for 20 h in the presence or absence of RS or heparin.

The experimental apparatus used for measurement of LDL was as described Previously (Cancel, L. M., et al., 2010, Atherosclerosis, 208(2):335-341; Cancel, L. M., et al., 2007, American Journal of Physiology: Heart and Circulatory Physiology, 293(1):H126-H132). The apparatus consists of eight Delrin® chambers, each connected to a laser excitation source and an emission detector. A Transwell filter containing the HCAEC monolayer was inserted and sealed within the transport chamber creating a luminal (top) and abluminal (bottom) compartment. At the beginning of each experiment DiI-LDL (5 mg/mL; Biomedical Technologies) was added to the luminal compartment. The fluorescent detection system was then used to measure the solute concentration in the abluminal compartment of each chamber as a function of time. Each transport experiment consisted of a one-hour equilibration period, followed by application of a 10 cm H₂O pressure differential and data collection for one hour. The permeability was calculated as

$${P}_{e} = \frac{{V}_{a}}{{AC}_{l}} \frac{Δ {C}_{a}}{Δ t}$$

where ΔCₐ/Δt is the change in the abluminal concentration with respect to time, Vₐ is the fluid volume in the abluminal compartment, Cₜ is the concentration in the luminal compartment, and A is the area of the filter.

Immunostaining of heparan sulfate. Monolayers of HCAEC grown in Transwell membranes were stained for heparan sulfate using mouse monoclonal anti-heparan sulfate antibody (10E4 epitope, 1:100 in 2% goat serum, AMSBIO) following previously established protocols (Zeng, Y., et al., 2012, PLOS One, 7(8):e43168). Briefly, monolayers were fixed in 2% paraformaldehyde and 0.1% glutaraldehyde in PBS for 30 min, blocked with 2% GS for 30 min, and incubated with primary antibody overnight at 4° C. in a humidified chamber. Monolayers were then rinsed and incubated with secondary antibody Alexa Fluor 488 goat anti-mouse IgG (1:300 in 2% GS) for one hour at room temperature, and counterstained with DAPI. The stained monolayers were imaged using a ZEISS LSM 510 confocal microscope. ImageJ was used to quantify image intensity. The coverage of heparan sulfate was analyzed using the methods described in previous work (Zeng, Y., et al., 2012, PLOS One, 7(8):e43168).

Preparation of subendothelial matrix samples. Monolayers of HCAECs were grown for 14 days in Transwell filters before the cells were removed by a method shown to leave intact subendothelial matrix attached to the Transwell (Pillarisetti, S., et al., 1997, Journal of Clinical Investigation, 100(4):867-874). Briefly, the monolayers were washed three times in PBS, incubated in 20 mM NH₄OH in 0.1% Triton X-100® for 5 min at room temperature, washed three times in PBS, and washed three times in basal media containing 3% BSA. The filters with subendothelial matrix were then incubated with RS for 24 h, and the permeability of the subendothelial matrix to DiI-LDL was measured.

Nuclear fractionation and extraction of proteins. Endothelial cells were maintained in MCDB-131 medium with 0.5% FBS for at least 18 h, then stimulated with TNF-α (10 ng/mL) for 30 minutes with or without RS pretreatment for 24 h (100-500 μg/mL). Tissue homogenates of endothelial cells were resuspended in a buffer, which consisted of 10 mM HEPES (pH 7.8), 15 mM KCl, 2 mM MgCl₂, 0.1 mM EDTA, 1 mM dithiothreitol (DTT), and 1 mM phenylmethylsulfonyl fluoride. After 10 min on ice, the tissue homogenates were pelleted and resuspended in two volumes of the buffer. Then, 3 M KCl was added dropwise to reach a 0.39 M KCl concentration. Nuclei were extracted from the cells with incubation for 1 h at 4° C. followed by centrifuged at 12,000 g for 1 h. The supernatants were then dialyzed in a buffer, which consisted of 50 mM HEPES (pH 7.8), 50 mM KCl, 0.1 mM EDTA, 1 mM DTT, and 1 mM phenylmethylsulfonyl fluoride with 10% (v/v) glycerol. The samples were then cleared by centrifugation and stored at −80° C. until needed for further use. Total protein concentration was determined by BCA (Thermo Fisher Scientific). The levels and phosphorylation of IκBα, IKKa and IKKβ in cytosolic fractions were measured by Western blotting as described below. p65 in the nuclear and cytosolic fractions was measured by Western blot studies. β-actin and histone H3 were used as controls for total protein in cytosolic or nuclear fractions, respectively. The activity of NF-κB in binding DNA was assessed in nuclear fractions by a DNA Binding TransAM NF-1Bp65 Assay (Active Motif).

Western blotting. Cells were lysed using RIPA lysis buffer with added protease and phosphatase inhibitors (10 μL/mL), EDTA (10 μL/mL), and phenylmethylsulfonyl fluoride (1 mM) (Thermo Fisher Scientific). They were sonicated for three minutes and then centrifuged at 10,000 g for 10 minutes to collect the total protein fraction. Concentration was determined using the Pierce BCA Protein Assay (Thermo Fisher Scientific). SDS PAGE were performed on the samples using the Invitrogen NuPAGE™ Bis-Tris protein gels. The proteins were transferred to nitrocellulose membranes using the wet transfer method (BioRad). The membranes were blocked with 4% StartingBlock™ T20 Blocking Buffer (Thermo Fisher Scientific) with 0.1% Tween-20® in PBS or TBS for one hour. The membranes were then incubated with the primary antibodies in 1% StartingBlock™ overnight at 4° C. (Table 2). The membranes were then incubated with HP-conjugated secondary antibodies (Cell Signaling) at 1:3500 dilution for two hours in 1% BLOTTO. SuperSignal™ West Femto ECL solution (Thermo Fisher Scientific) was used to detect the antibodies and imaging was performed on a G:BOX imaging system (Syngene, Inc.).

TABLE 2
Primary antibodies used for immunoblotting.

| | | | | Dilution |
|---|---|---|---|---|
| Target Protein | Company | Catalog # | Species/Isotype | Ratio |
| GAPDH | Cell Signaling | 2118S | anti-rabbit | 1:500 |
| α-SMA | Abcam | ab21027 | anti-rabbit | 1:500 |
| α-SMA (Cy3) | Sigma-Aldrich | C6198 | anti-mouse | 1:200 |
| α-tubulin | Sigma-Aldrich | T9026 | anti-mouse | 1:500 |
| β-actin | Santa Cruz Biotech | sc-47778 | anti-mouse | 1:500 |
| eNOS | Santa Cruz Biotech | sc-654 | anti-rabbit | 1:500 |
| F4/80 | Abcam | ab6640 | anti-mouse | 1:500 |
| FAK | Cell Signaling | 3285S | anti-rabbit | 1:100 |
| Histone H3 | Cell Signaling | 9715S | anti-mouse | 1:500 |
| IκBα | Cell Signaling | 4814S | anti-rabbit | 1:500 |
| IKKα | Santa Cruz Biotech | sc-7606 | anti-mouse | 1:500 |
| IKKβ | Cell Signaling | 2678S | anti-rabbit | 1:500 |
| NF-κBp65 | Thermo Fisher | PA1186 | anti-rabbit | 1:500 |
| | Scientific | | | |
| PECAM-1 | Cell Signaling | 3528S | anti-mouse | 1:500 |
| Phospho-IκBα | Thermo Fisher | MA515224 | anti-rabbit | 1:500 |
| (S32/34) | Scientific | | | |
| Phospho-IKKα/β | Cell Signaling | 2797S | anti-rabbit | 1:500 |
| TKS5 | Sigma-Aldrich | 09-403 | anti-rabbit | 1:100 |

Murine model of atherosclerosis. Male and female ApoE−/− mice (B6.129P2-Apoetm1Unc/J) were used for this study (Jackson Laboratories, Inc.). The animals were given high fat chow which was the standard formulation Clinton/Cybulsky high fat rodent diet with regular casein and 1.25% added cholesterol (D12108C; Research Diets, Inc.). For RS treated animals, the high fat diet (HFD) was formulated with 0.75 g of RS per kg diet. At 12 weeks of age, the mice were switched from normal chow to high fat chow for 4 weeks. The HFD group remained on this diet for an additional 9 weeks while the treatment group was switched to high fat chow with RS. At 4 and 12 weeks after start of HFD, the vessels of the mice were imaged using high resolution ultrasound as described below. At 13 weeks of HFD, the mice were sacrificed and the blood, heart, aorta, liver, and white adipose tissues were harvested for further analysis. For control, male and female wild type mice (n=5) were fed a standard diet for 13 weeks.

High resolution ultrasound imaging. High resolution ultrasound was performed using the Visual Sonics VEVO 2100 system with the MS500D transducer in the ApoE−/− model. The mice were anesthetized with isoflurane and body temperature monitored using a probe. Prior to imaging, the fur on the chest was removed using Nair™. First, the longitudinal section of the aortic arch was located using the B-mode option. Pulsed wave Doppler and 3D echocardiography images were then obtained for calculating peak systolic velocity (PSV), end diastolic velocity (EDV), and mean velocity (MV). The longitudinal section of the left common carotid artery was located using B-mode and pulsed wave Doppler images were obtained for the same parameters in the carotid artery. Finally, the cross section of the aortic arch was imaged in B-mode and color Doppler. Diameter measurements of the aortic arch determined from cross sections were used to calculate circumferential strain using the following formula:

$$\frac{1}{2} ( {( \frac{Sys}{Dia} )}^{2} - 1 ) \times 100$$

where Sys and Dia are the peak systolic and end diastolic diameters, respectively.

Histological staining. Aortic roots at optimal cutting temperature were embedded in NEG-50™ and cut into 7 μm thick sections using a cryostat (CM1510 S; Leica). These sections were stained with Oil Red 0/hematoxylin to measure lipid depot using the protocol below. Individual lesion area was determined by averaging the maximal values. For the liver samples, the tissues were placed in NEG50™ embedding medium and then frozen in liquid nitrogen cooled isopentane. The samples were serially sectioned to create 7 μm thick cryosections and Oil Red-O staining was performed.

For the fat tissues, white adipose tissues (gonadal and inguinal depots) were fixed overnight in 4% formaldehyde and paraffin sections were created using standard methods. The size of adipocytes in white adipose tissues were quantified in H&E staining using Image J software.

For liver tissues, the degree of NAFLD was determined by H&E staining paraffin-embedded liver sections (4 μm thick). The sections were evaluated by a single-blinded clinical pathologist and NAFLD Activity Scores (NAS) were determined using the protocol below.

Oil Red-O staining. A stock Oil Red O (Electron Microscopy Sciences) solution was made with 3 mg/mL Oil Red O in 99% isopropanol. The stock was diluted in a 3:2 ratio in ultrapure water. Frozen cryosections were air dried and then fixed in 10% formalin. They were stained with Oil Red O and counterstained with Mayer's hematoxylin (Electron Microscopy Sciences). The sections were mounted with aqueous mounting medium for imaging (Vector Labs).

Immunostaining for tissues. Lesional macrophages (F4/80), vSMCs (α-SMA), PECAM-1, eNOS, IκBα and p-p65 were detected by immunoperoxidase or immunofluorescence (Table 2). Positive staining was expressed as percentage of total plaque area or number of positive cells per lesion area. In each experiment, negative controls without the primary antibody or using a nonrelated antibody were included to check for nonspecific staining.

En face imaging of aorta. Atherosclerotic lesions were quantified by en face analysis of the whole aorta. For en face preparations, the aorta was opened longitudinally, from the heart to the iliac arteries, while still attached to the heart and major branching arteries in the body. The aorta from the heart to the iliac bifurcation was then removed and was pinned out on a white wax surface in a dissecting pan using stainless steel pins 0.2 mm in diameter. After overnight fixation with 4% paraformaldehyde and a rinse in PBS, the aortas were immersed for 6 min in a filtered solution containing 0.5% Oil Red-O, 35% ethanol and 50% acetone and destained in 80% ethanol. The Oil Red-O-stained aortas were photographed, and the atherosclerotic lesions were quantified using IP Win32 v4.5 software.

Pharmacokinetics of rhamnan sulfate in vivo. To test for oral bioavailability of RS, male mice (C57BL/6; Jackson Labs) were given FITC-conjugated RS (RS-FITC) (0.25 g RS/kg mouse) though oral gavage. Mice were sacrificed at the time points of 0, 1, 4, 12 and 24 hours (n=2) and blood, aorta, liver, and heart were harvested. The tissue samples were lysed and RS-FITC concentration measured by reading fluorescence intensity with correction for background fluorescence. A calibration curve was made for RS-FITC in each tissue to convert fluorescence to concentration.

To measure the half-life of RS in the blood a mouse was injected intravenously via the tail vein with 0.5 mg of RS-FITC. A 10 μL blood sample was collected from the saphenous vein 1 minute after injection, and then every five minutes for 25 minutes. The blood samples were centrifuged at 12,000 rpm for 5 minutes and the plasma was used for fluorescence measurements. Fluorescence intensity was measured using a plate reader (BioTek). A calibration curve for RS-FITC in mouse plasma was created. The plasma concentration data was fitted to an exponential curve (first order elimination process is assumed). Extrapolating from the resulting equation the concentration at t=0 is 0.278 mg/mL. The apparent volume of distribution, VD, is calculated from the relationship VD=Dose/Cp⁰, where Cp⁰ is the concentration at t=0. VD was calculated to be 1.8 mL, approximately equal to the blood volume (the blood volume of a mouse is 77-80 mL/kg). The half-life was calculated from the elimination rate constant

$$( {t}_{1 / 2} = \frac{ln 2}{k} ) .$$

With the data above, (k=0.0328 min⁻¹) the half-life is 21.1 minutes. Extrapolating from the data, the concentration drops below the lowest effective concentration tested (1 mg/mL) after 170 minutes.

Raman spectroscopy. Raman imaging was performed using a custom built 830 nm confocal Raman microscope with a 60× water immersion objective (NA=1.2; Olympus) (Feng, X., et al., 2017, Biomedical Optics Express, 8(6):2835-2850). The scattered light was collected by a spectrograph and a CCD camera though a 50 μm core diameter fiber, which also acts as the pinhole of the confocal system. Fresh sections with 10 μm thickness were mounted on low-background Raman substrates (magnesium fluoride window or quartz slide) for Raman imaging. For every animal, one liver section was prepared, and 2-5 regions of interest were randomly selected from each section. Raman imaging was performed with a 0.25 or 0.5 s integration time and a step size of 0.75 or 1 Image size varied from 24×24 μm² to 40×40 μm². Data preprocessing was performed using MATLAB® (R2017a, MathWorks). Preprocessing steps included wavenumber calibration, background removal, cosmic ray removal, smoothing, and fluorescence background removal. Spectra were normalized to the area under the curve between 600 and 1800 cm⁻¹. Cluster analysis was performed for each image by k-means algorithm (Kochan, K., et al., 2015, Journal of Biophotonics, 8(7):597-609). The first 100 principal components accounting for 95%-99% of the variation in the data set served as the input for the k-means. Each image contained at most three clusters, annotated as the lipid-rich zone, protein-rich zone, and others. Only the spectra within the lipid-rich zone were extracted to calculate the degree of unsaturation. The average Raman spectrum of the lipid-rich zone was compared between different groups. The degree of unsaturation was calculated by the peak ratio of 1656 cm⁻¹ (integrated from 1645-1675 cm⁻¹) to 1441 cm⁻¹ (integrated from 1420-1480 cm⁻¹), which corresponds to the ratio of C=C stretching vibration and the band related to the CH₂ scissoring mode (Kochan, K., et al., 2015, Journal of Biophotonics, 8(7):597-609; Kochan, K., et al., 2013, Analyst, 138(14):3885-3890).

Preparation of rhamnan sulfate and heparin biochip. Biotinylated RS or heparin was prepared by conjugating the reducing end to amine-PEG3-biotin (Pierce) (Kim, S. Y., et al., 2018, Journal of Biological Chemistry, 293(40):15381-15396). In brief, RS or heparin (2 mg) and amine-PEG3-biotin (2 mg, Pierce) were dissolved in 200 μL H₂O and 10 mg NaCNBH₃ was added. The reaction mixture was heated at 70° C. for 24 h, after that a further 10 mg NaCNBH₃ was added, and the reaction was heated at 70° C. for another 24 h. After cooling to room temperature, the mixture was desalted with a spin column (3,000 MWCO). Biotinylated RS or heparin was collected, freeze-dried, and used for surface plasmon resonance (SPR) chip preparation. The biotinylated RS or heparin was immobilized to a streptavidin chip (GE Healthcare) based on the manufacturer's protocol. Successful immobilization of RS or heparin was confirmed by the observation of an about 100 resonance unit (RU) increase on the sensor surface after 20 μL injection of RS or heparin (1 mg/mL). The control flow cell was prepared by 1 min injection with saturated biotin.

Surface plasmon resonance of binding between rhamnan sulfate and proteins. Recombinant human FGF-1 and FGF-2 were a gift from Amgen. Human antithrombin III (AT) (Hyphen Biomed), recombinant human platelet-derived growth factor (PDGF-BB), NF-κB p50, and NF-κB p65 (Abcam) were purchased. The interactions between RS and proteins were measured using the BIAcore™ 3000 SPR system (GE Healthcare). The protein samples were diluted in HBS-EP buffer (0.01 M HEPES, 0.15 M NaCl, 3 mM EDTA, 0.005% surfactant P20, pH 7.4). Different dilutions of protein samples were injected at a flow rate of 30 μL/min. At the end of the sample injection, HBS-EP buffer was flowed over the sensor surface to facilitate dissociation. After a 3 min dissociation time, 30 μL of 2 M NaCl was injected to fully regenerate the surface. The response was monitored as a function of time (sensorgram) at 25° C.

NAFLD activity score analysis. The NAS was calculated for each liver sample as described previously (Kleiner, D. E., et al., 2005, Hepatology, 41(6):1313-1321). Briefly, the score is the sum of the scores for steatosis (0-3), lobular inflammation (0-3), and ballooning (0.2), with an overall score range of 0-8. Steatosis score was also assessed to grade the percentage involvement by steatotic hepatocytes as follows: grade 0, 0-5%; grade 1, >5-33%; grade 2, >33-66%; grade 3, >66%. In addition, Brunt's histological scoring system was used to evaluate the degree of hepatocellular ballooning and lobular inflammation (Brunt, E. M., et al., 1999, The American Journal of Gastroenterology, 94(9):2467-2474). The degree of lobular inflammation was measured, numbering the inflammatory foci per 200× field as follows: grade 0, no foci; grade 1, 1-2 foci; grade 2, 2-4 foci; grade 3, 5+foci. The degree of hepatocellular ballooning was assessed according to the presence of balloon hepatocytes as follows: grade 0, none, grade 1, few balloon cells; grade 2, many balloon cells/prominent ballooning. Minimal criteria for steatohepatitis (NASH) included the combined presence of grade 1 steatosis, grade 1 hepatocellular ballooning, and grade 1 lobular inflammation.

Statistical analysis. All results are shown as mean±standard error of the mean. Comparisons between only two groups were performed using a 2-tailed Student's t-test. Multiple comparisons between groups were analyzed by 2-way ANOVA followed by a Tukey post-hoc test. A 2-tailed probability value p<0.05 was considered statistically significant.

What is claimed is:

1. A composition for treating or preventing a metabolic disease or disorder or a circadian rhythm-related disorder comprising rhamnan sulfate, wherein the rhamnan sulfate has a molecular weight of about 100-200 kDa.

2. The composition of claim 1, wherein the rhamnan sulfate has a molecular weight of about 150 kDa.

3. A method of treating or preventing a metabolic disease or disorder in a subject in need comprising administering to the subject a composition comprising a therapeutically effective amount of rhamnan sulfate.

4. The method of claim 3, wherein the metabolic disease or disorder is non-alcoholic fatty liver disease (NAFLD).

5. The method of claim 4, wherein non-alcoholic fatty liver disease (NAFLD), through modification of circadian rhythm pathways, is treated or prevented by preventing or treating the effects of sleep disturbances or disorders.

6. The method of claim 4, wherein the rhamnan sulfate has a molecular weight of about 100-200 kDa.

7. The method of claim 5, wherein the rhamnan sulfate has a molecular weight of about 150 kDa.

8. A method of treating or preventing a disease or disorder associated with a circadian rhythm gene or a circadian rhythm-related gene in a subject comprising administering to the subject a composition comprising a therapeutically effective amount of rhamnan sulfate.

9. The method of claim 8, wherein the administering of a therapeutic amount of rhamnan sulfate treats or prevents sleep deprivation.

10. The method of claim 9, wherein the sleep deprivation results from one or more selected from the group consisting of sleep disorders, depression, anxiety, traumatic brain injuries, post-traumatic stress disorder, work-shift disorders, insomnia, and neurological disorders.

11. The method of claim 8, wherein the circadian rhythm gene or circadian rhythm-related gene is one or more selected from the group consisting of PER3, TPPP, DBP, and WEE1.

12. The method of claim 11, wherein the rhamnan sulfate has a molecular weight of about 100-200 kDa.

13. The method of claim 12, wherein the rhamnan sulfate has a molecular weight of about 150 kDa.
